(19) United States
(12) Patent Application Publication
(10) Pub. No.: US 2022/0213552 A1
(43) Pub. Date: Jul. 7, 2022

(54) METHODS FOR DIAGNOSIS AND PROGNOSIS OF PROSTATE CANCER

(71) Applicant: Phenotype Diagnostics AB, Umeå (SE)

(72) Inventors: Pernilla Wikström, Umeå (SE); Anders Bergh, Umeå (SE); Elin Thysell, Umeå (SE)

(21) Appl. No.: 17/432,642

(22) PCT Filed: Feb. 21, 2020
(86) PCT No.: PCT/EP2020/054681

§ 371 Date: Aug. 20, 2021

(30) Foreign Application Priority Data
Feb. 22, 2019 (SE) 1950232-7

(51) Int. Cl. C12Q 1/6886; G01N 33/574; A61K 45/06

(52) U.S. Cl. CPC C12Q 1/6886; G01N 33/57434; C12Q 2600/156; C12Q 2600/112; A61K 45/06

(57) ABSTRACT

The invention relates to a method for determining tumor aggressiveness in a subject diagnosed with prostate cancer and having a tumor, said method comprising (a) evaluating the level of cell differentiation in a sample comprising tumor-derived material from the said subject; and (b) evaluating the level of proliferating cells in the said sample. In a further aspect, the invention comprises determining the ratio between the level of proliferating cells and the level of cell differentiation and in the sample. The invention further relates to methods for determining the need of curative treatment in a subject diagnosed with prostate cancer, as well as to methods for treating prostate cancer in a subject in need thereof.

### TECHNICAL FIELD

The invention relates to a method for determining tumor aggressiveness in a subject diagnosed with prostate cancer and having a tumor, said method comprising (a) evaluating the level of cell differentiation in a sample comprising tumor-derived material from the said subject; and (b) evaluating the level of proliferating cells in the said sample. In a further aspect, the invention comprises determining the ratio between the level of proliferating cells and the level of cell differentiation in the sample. The invention further relates to methods for determining the need of curative treatment in a subject diagnosed with prostate cancer, as well as to methods for treating prostate cancer in a subject in need thereof.

### BACKGROUND ART

Bone metastatic disease is the lethal end-stage of aggressive prostate cancer. Patients with metastatic prostate cancer are generally treated with androgen deprivation therapy (ADT). This initially reduces metastasis growth, but after some time castration resistant PC prostate cancer (CRPC) develops. Although several new treatments for CRPC have become available they only temporarily retard disease progression (1). Therapy-selection in individual patients as well as future therapeutic developments need to be guided by deeper understanding of bone metastasis biology. This can probably not be obtained by studying primary tumors only or metastases at other locations, since metastases phenotypically diverge due to clonal expansions under the profound influence of different micro-environments, resulting in site-dependent responses to treatment (2, 3).

From studies of the transcriptome and proteome of bone metastases from patients, marked differences between metastases and primary tumors have been identified. Furthermore, metastasis subgroups of apparent biological importance have been identified (4-9). Based on gene expression of canonically AR-regulated genes, 80% of the examined prostate cancer bone metastases were defined as AR driven and 20% were defined as non-AR-driven (7). AR-driven bone metastases had high sterol biosynthesis, amino acid and fatty acid degradation, and nucleotide biosynthesis (7), while non-androgen driven metastases showed high immune cell (7) and bone cell activities (8). Proteomic analysis identified two molecular subtypes of bone metastases with different phenotypes and prognosis (9). These observations suggest possibilities for subtype-related treatment of bone metastatic prostate cancer.

High proliferation and low tumor cell PSA synthesis in primary tumors have been linked to poor prognosis (11, 12, 31-33), but have not previously been combined for prognostication. WO 2006/091776 relates to the identification and use of gene expression profiles with clinical relevance to prostate cancer. The invention involves the use of expression levels of a set of 41 genes. It is disclosed that the level of expression of MIB1 (Ki67) was higher in metastatic small cell prostate cancer than in localized prostate cancer and that the level of expression of PSA was higher in localized than in metastatic small cell cancers.

However, there is a need for improved methods for determining tumor aggressiveness in subjects diagnosed with prostatic cancer.

### BRIEF DESCRIPTION OF THE DRAWINGS

FIG. 1. Principal component analysis (PCA) and unsupervised clustering of 72 bone metastasis samples, based on whole genome expression analysis (Illumina bead chip array) identifies three main clusters of samples; MetA, MetB, and MetC. Score plot (a) and loading plot (b) showing MetA-C in black, dark gray and light gray, respectively, based the two first principal components and the clusters in (c). Samples from castration-resistant prostate cancer (CRPC) patients are represented by circles and samples from non-treated and short-term castrated patients are shown as squares. Two neuroendocrine metastases are indicated by stars. Selected sets of gene products enriched in the different metastasis clusters are highlighted. d) Predictions of non-treated, short-term treated, and neuroendocrine samples (gray squares) into clusters defined from PCA analysis of CRPC samples only e) Kaplan-Maier plot showing poor cancer-specific survival for MetB patients after androgen-deprivation therapy (ADT) and f) Top four enriched network categories per metastasis subtype, according to gene set enrichment analysis using the MetaCore software.

FIG. 2. Consistency of metastasis clusters based on the two first principal components for the PCA analysis using five clustering algorithms: i) Hierarchical clustering using the Euclidian distance and Ward linkage, ii) Hierarchical clustering using the Manhattan distance and Ward linkage, iii) k-means clustering, iv) Self Organizing maps and v) Affinity propagation.

FIG. 3. Principal component analysis and orthogonal projections to latent structures discriminant analysis (OPLS-DA) of bone metastasis samples, based on gene expression levels of top 60 differentiating genes for each subtype (see Table 1) showing the score plot, loading plot and hierarchical clusters of a-c) GEO Datasets GSE29650 and GSE101607 and d-f) RNA seq. data (52), g-h) OPLS-DA model for MetA-C based on 72 samples (GSE29650 and GSE101607) and prediction of 43 external samples (yellow) (50), giving frequencies as shown in Table 1.

FIG. 4. Representative tissue sections of MetA, MetB and Met C bone metastases and associated primary tumors stained with HTX-eosin (a-c) and (j-l), PSA (d-f) and (m-o), and Ki67 (g-i) and (p-r). MetA is characterized by moderate cellular atypia, glandular differentiation, relatively low fraction of Ki67 positive cells (proliferating cells) and high PSA immunoreactivity (IR). MetB shows prominent cellular atypia, lack of glandular differentiation, low PSA IR and high tumor cell proliferation. MetC shows prominent cellular atypia with glandular differentiation detectable in some cases, low cell proliferation, relatively low tissue PSA IR, and relatively high stroma/epithelial ratio. MetA associated primary tumors are characterized by high PSA IR and relatively low proliferation. MetB associated primary tumors show low PSA IR, high proliferation, and a reactive stroma response. MetC associated primary tumors show relatively high proliferation with PSA IR and reactive stroma response intermediate between MetA and MetB cases. Bar indicates 100 μm.

FIG. 5. Kaplan-Meier analysis of PSA immunoreactivity (IR) score and proliferation rate (fraction of Ki67-stained tumor cells) in metastasis samples in relation to cancer-specific survival after treatment with androgen-deprivation therapy (ADT). PSA IR was dichotomized as above (high) or below (low) median and Ki67 as quartile 4 (high) or below (low) (a-b). A combinatory PSA and Ki67 score was obtained based on their inverse correlation and the cutoffs used in a-b (c) Patients with high PSA, low Ki67 metastasis IR show the best prognosis with significantly longer cancer-specific survival after first ADT than other patients (d).

FIG. 6. Paired observations of androgen receptor (AR) (a), PSA (b) and Ki67 (c) immunoreactivity (IR) scores in bone metastases of subtypes A-C and in corresponding primary tumor biopsies. The AR and PSA IR were significantly reduced and the proliferation (fraction of Ki67 positive tumor cells) significantly increased in MetA metastases compared to their matched primary tumors.

FIG. 7. Kaplan-Meier analysis of combinatory PSA and Ki67 immunoreactivity (IR) in primary tumor samples in relation to cancer-specific survival after treatment with androgen deprivation therapy (ADT) in metastatic MetA-C patient cohort (a) and in a validation cohort of TUR-P diagnosed patients (b). PSA IR was dichotomized as above (high) or below (low) median and Ki67 as quartile 4 (high) or below (low), using cut-off values for the corresponding cohort. a) Patients with high PSA, low Ki67 primary tumor IR show significantly longer cancer-specific survival after first ADT than other patients. b) Patients with high PSA, low Ki67 show longer and patients with low PSA, high Ki67 show shorter cancer-specific survival after first ADT compared to other patients. c-d) Multivariate Cox analysis shows that the combinatory PSA, Ki67 IR scores evaluated in primary tumors add prognostic value to Gleason scores in metastatic (c) and TUR-P (d) patient cohorts.

FIG. 8. Kaplan-Meier survival analysis of PSA immunoreactivity (IR) (a-b) and a combinatory immunoreactivity (IR) score for PSA and Ki67 (c-f) in relation to cancer-specific survival of patients diagnosed at TUR-P and managed by watchful-waiting. a, c, e) All patients in the cohort and b, d, f) Patients diagnosed with GS≤6 tumors. PSA IR was dichotomized by the median value 9 as high (IR=12) or low (<12). Ki67 was dichotomized by cut-off value for the median (c, d) as Ki67 med-high (Ki67≥2.7%) or Ki67 med-low (<2.7%) or the highest quartile (e, f) as Ki67 Q4-high (Ki67≥5.4%) or Ki67 Q4-low (<5.4%).

FIG. 9. Sensitivity (black) and specificity (grey) of Ki67 (a) and PSA (b) tumor immunoreactivity in identifying death from prostate cancer at different cut-off scores. Patients were diagnosed at TUR-P (1975-1991) and managed by watchful waiting. Median (PSA and Ki67) and Q4 (Ki67) levels for the TUR-P cohort are indicated. The −log (P) values for Cox regression survival analysis using the indicated cut-off values are given in grey.

FIG. 10. PSA immunoreactivity score (A) and fraction of Ki67 positive tumor cells (B) were evaluated in primary tumor biopsies from prostate cancer patients within different risk groups; low to intermediate (n=45), high to locally advanced (n=21), regionally metastasized (n=16), and peripherally metastasized (n=28) (consecutive UCAN cohort, treated 2013-2015, for risk definition see experimental methods). Figure confirms decreased PSA and increased Ki67 immunoreactivity with disease progression.

FIG. 11. Sensitivity and specificity of Ki67 (A) and PSA (B) immunoreactivity for differentiation of patients with indolent disease (low to intermediate risk tumors, n=45) from patients in need of therapy for progressive disease (high risk, locally advanced, regionally metastasized or peripherally metastasized, n=65). For specific cut-off values, see Table 7 and 8.

FIG. 12. Sensitivity and specificity of Ki67 (A) and PSA (B) immunoreactivity for differentiation of patients with metastatic disease according to short or long time to progression after androgen-deprivation therapy. For specific cut-off values, see Table 9 and 10.

FIG. 13. Kaplan-Meier analysis of combinatory immunoreactivity score in primary tumor biopsies for PSA and Ki67 in relation to time to disease progression (A) and in TURP tissue in relation to cancer-specific survival (B) in patients treated with androgen-deprivation therapy due to metastatic disease. PSA was dichotomized by the value 8 as high (>8) or low (≤8). Ki67 was dichotomized by cut-off value 16% as high (≥16) or low (<16).

FIG. 14. A) Sensitivity and specificity of the Ki67/PSA immunoreactivity ratio for identifying death from prostate cancer at different cut-offs. Patients were diagnosed at TUR-P (1975-1991) and managed by watchful waiting. The Ki67/PSA-ratio was obtained by dividing fraction of Ki67 positive tumor cells with the corresponding PSA immunoreactivity score. For specific cut-off values, see Table 11. B) Kaplan-Meier survival analysis of Ki67/PSA immunoreactivity ratio in relation to cancer-specific survival of patients diagnosed at TUR-P and managed by watchful-waiting when dichotomized based on Ki67/PSA=0.2.

FIG. 15. Sensitivity and specificity of the Ki67/PSA immunoreactivity ratio for differentiating patients with indolent disease (low to intermediate risk tumors, n=45) from patients in need of therapy for progressive disease (high risk, locally advanced, regionally metastasized or peripherally metastasized, n=65). For specific cut-off values, see Table 13. The Ki67/PSA-ratio was obtained by dividing fraction of Ki67 positive tumor cells with the corresponding PSA immunoreactivity score.

FIG. 16. A) Sensitivity and specificity of the Ki67/PSA immunoreactivity ratio for differentiation of patients with metastatic disease according to short or long time to progression after androgen-deprivation therapy. For specific cut-off values, see Table 14. B) Kaplan-Meier survival analysis of Ki67/PSA immunoreactivity ratio in primary tumor biopsies in relation to time to progression after androgen-deprivation therapy when dichotomized based on Ki67/PSA=2.1. C) Kaplan-Meier survival analysis of Ki67/PSA immunoreactivity ratio in bone metastasis tissue in relation to time to progression after androgen-deprivation therapy when dichotomized based on Ki67/PSA=2.1.

### DESCRIPTION OF THE INVENTION

Three molecular subtypes of prostate cancer bone metastases, named MetA, MetB and MetC, have been identified. The said subtypes are related not only to disease outcome, but also to morphology and phenotypic characteristics, and are suggested to be of high clinical significance. Treatment naive and CRPC metastases are found within all subtypes, suggesting that factors other than hormone treatment history are key determinants of subgroup identity. The clinically most contrasting subtypes, MetA and MetB, show characteristics similar to the two subgroups BM1 and BM2, respectively, recently identified by proteome profiling of metastasis samples (9). Furthermore, MetA-C show features resembling subtypes recently described for localized prostate tumors; prostate cancer subtype 1-3 (PCS1-3) (18) and luminal A, luminal B and basal subtypes as determined by the PAM50 breast cancer test (19). Importantly, however, the top 180 differentiating gene products for MetA-C. The functionally enriched gene products, show a minor overlap with the biomarkers suggested to differentiate primary tumors into molecular subtypes (18, 19) and with biomarkers on approved tests for predicting risk and selecting therapy in patients with localized disease (Prolaris, OncotypeDx, GenomeDx) (51), with a total overlap of 46/180 gene products (25%). Based on analysis of MetA-C-associated gene transcripts, the MetA-C subtypes were predicted in an external validation cohort (50) at frequencies comparable to those originally observed.

The most common metastasis subtype (MetA) seems to be of luminal cell origin, according to expression of luminal cell differentiation markers and androgen-stimulated genes, including KRT18, FOXA1 and KLK3 (PSA), and signs of glandular differentiation. MetA patients have high serum PSA levels and show good prognosis after ADT. The phenotype of MetA thus resembles that of luminal prostate epithelium.

The MetB subtype shows some features similar to neuroendocrine tumors, such as low AR signaling and high cell cycle and DNA damage response (20), but chromogranin expression is generally low and KRT18 expression retained, suggesting luminal dedifferentiation. The contrasting processes of cell differentiation and proliferation are both driven by androgens in the prostate (21-23), but in a context dependent way that seems reprogrammed during cancer progression by coactivators and corepressors modulating the AR cistrome (24, 25). AR activation in the presence of coactivator FOXA1 results in cell differentiation, PSA secretion and suppressed proliferation (21-23, 26), while in cells with low FOXA1 this instead stimulates cell proliferation (23). In the MetB subtype, androgen-stimulated gene expression is generally low, tumor cells are dedifferentiated, and cell proliferation is high, in parallel with transcript levels of the proliferation-associated transcription factor FOXM1. FOXM1 is known to initiate mitosis (17) and FOXM1 inhibition has been shown to retard tumor growth in a model system for the PCS1 subtype (27).

In the current study, approximately 15% of the samples showed an intermediate subtype with characteristics of both MetA and MetB and in the external cohort (50) this was observed in about 9%. In the LNCaP cell line with a general gene expression pattern similar to PCS2 primary tumors (18), single cell sequencing has demonstrated the existence of multiple sub-clones where some appear similar to MetA whereas others are more MetB-like with high cell proliferation and reduced androgen dependency (28). Collectively, this suggest that the luminal-derived MetA subtype may be able to dedifferentiate in to the more aggressive MetB subtype, possibly driven by altered expression of AR co-regulators such as FOXA1 and/or FOXM1.

The relatively uncommon subgroup MetC is identified based on enrichment of transcripts involved in stroma-epithelial interactions such as cell adhesion, cell and tissue remodeling, immune responses and inflammation. Processes in MetC thus resembles those previously described by us for non-AR-driven bone metastases (7, 8) and for PCS3/basal-like primary tumors of presumed basal cell origin (18, 19). One suggested upstream regulator of MetC is the C/EBP transcription factor, generally associated with inflammation and down-regulated by AR signaling (29). C/EBP is anti-apoptotic and causes chemo-resistance in CRPC, and thus constitutes a potential therapeutic target (29). The stroma fraction in MetC is higher than in MetA and, although this is repeatedly observed in separate metastases of MetC patients, it remains to be shown to what extent the molecular characteristics of MetC is a consequence of lower epithelial content or a key marker of a clearly different tumor phenotype. Furthermore, the cellular origin of MetC and surrogate markers for this apparently multi-faced metastasis phenotype remains to be discovered.

Apparently, the MetA-C subtypes can be determined by other means than by complex molecular profiling. MetB and corresponding primary prostate biopsies are characterized by tumor cell proliferation and dedifferentiation, easily identified by high Ki67 and low PSA immunostaining or by high MCM and low PSA, as recently suggested for BM2 (9). This markedly aggressive phenotype could thus probably be suspected simply by analyzing few surrogate markers, similarly to what is regularly done in breast cancer (30). High proliferation and low tumor cell PSA synthesis in primary tumors have been linked to poor prognosis (11, 12, 31-33), but have not previously been combined for prognostication.

When molecular drivers for different metastasis subtypes have been defined, subtype-related treatments could be developed. If androgen signaling promote cell differentiation and inhibit proliferation in subsets of metastases, as could be the case in MetA, ADT may in some cases have adverse effects and additional metabolic targeting could be an option. In other cases, such as MetB patients, ADT should probably be complemented upfront with i.e. chemotherapy, or by direct targeting of to tumor promoting factors driving the cell cycle or DNA repair. Patients with MetB bone metastases have reduced AR levels and morphological signs of a reactive stroma response already in their primary tumor stroma, something that has been previously associated with poor response to ADT and poor prognosis (15). For those cases, stroma targeted therapies could be of interest. In breast cancer, responsiveness to hormonal therapy seems to be regulated by signals in the cancer stroma as stroma interfering was able to convert basal, hormone treatment-resistant breast cancer into a luminal, treatment-responsive subtype (34, 35). For MetC patients, potential therapeutic targets in the tumor micro-environment may already be available, such as immune and bone cells.

In conclusion, bone metastases in prostate cancer patients can be separated into at least three molecular subtypes with different morphology, phenotype and outcome. Those subtypes may benefit from different treatments and can be identified by analyzing surrogate markers in metastases, in primary tumors and possibly in liquid biopsies mirroring the whole tumor burden in a patient.

Consequently, in a first aspect the invention provides a method for determining tumor aggressiveness in a subject diagnosed with prostate cancer and having a tumor, said method comprising:

(a) evaluating the level of cell differentiation in a sample comprising tumor-derived material from the said subject; and

(b) evaluating the level of proliferating cells in the said sample;

wherein

(i) a low level of cell differentiation and a high level of proliferating cells are associated with high tumor aggressiveness, and

(ii) a high level of cell differentiation and a low level of proliferating cells are associated with low or moderate tumor aggressiveness.

The term “tumor-derived material” means a material which comprises tumor cells or derivatives thereof. Preferably, the tumor-derived material consists of, or comprises, tumor cells. However, tumor-derived material also includes RNA and protein. The tumor-derived material can preferably be derived from the tumor as such. Alternatively, tumor-derived material can be derived from blood or urine from a subject having a tumor. The said tumor can be a primary tumor or a metastasis, such as a bone metastasis.

The term “sample” means matter that is gathered from the body with the purpose to aid in the process of a medical diagnosis and/or evaluation of an indication for treatment, further medical tests or other procedures. The said sample is preferably obtained by biopsy. A “biopsy” is a medical test involving extraction of sample cells or tissues for examination to determine the presence or extent of a disease. The sample can e.g. be analyzed chemically and/or examined under a microscope. The biopsy can e.g. be an incisional biopsy wherein a portion of abnormal tissue is extracted without removing the entire lesion or tumor. Alternatively, the biopsy can be e.g. a liquid biopsy where tumor-derived material is obtained from a blood or urine sample.

In one embodiment, the said method comprises:

(a) evaluating the level of cell differentiation in a sample comprising tumor-derived material from the said subject;

(b) evaluating the level of proliferating cells in the said sample; and

(c) deciding reference cut-off values for the level of cell differentiation and the level of proliferating cells;

wherein
(i) a lower level of cell differentiation compared to the corresponding reference cut-off value and a higher level of proliferating cells compared to the corresponding reference cut-off value are associated with high tumor aggressiveness, and
(ii) a higher level of cell differentiation compared to the corresponding reference cut-off value and a lower level of proliferating cells compared to the corresponding reference cut-off value are associated with low or moderate tumor aggressiveness.

Preferably, evaluating the level of cell differentiation in the sample comprises evaluating the level of prostate-specific antigen (PSA), e.g. by evaluating PSA immunoreactivity and determining the “PSA immunoreactivity score”. In the present context, the term “PSA immunoreactivity score” (or “PSA score”) means the score which is obtained by determining the percentage (0=No staining, 1=1-25%, 2=26-50%, 3=51-75% and 4=76-100% of tumor epithelial cells stained) and intensity (0=No staining; 1=weak; 2=moderate; and 3=intense) of immunostained tumor epithelial cells. The immunoreactivity score is obtained by multiplying the scores for distribution and intensity, as previously described (10), giving scores in the range of 0-12.

Preferably, evaluating the level of proliferating cells in the sample comprises evaluating the fraction of Ki67, Proliferating Cell Nuclear Antigen (PCNA), or MCM positive cells. Evaluating the level of proliferating cells preferably comprises evaluating Ki67 immunoreactivity. Preferably, Ki67 immunoreactivity can be quantified as the percentage of stained tumor epithelial cells as previously described (10).

In a further aspect, the invention provides a method comprising deriving a proliferation-PSA combination score from the proliferation/PSA ratio, said proliferation/PSA ratio being calculated by dividing the fraction of proliferating tumor cells in the sample multiplied with 100 with the PSA immunoreactivity score in the sample. In other words, the proliferation/PSA ratio is calculated by dividing the percentage of proliferating tumor cells in the sample with the PSA immunoreactivity score in the sample. According to this method, a high proliferation-PSA combination score is associated with high tumor aggressiveness, and a low proliferation-PSA combination score is associated with low or moderate tumor aggressiveness.

Examples of combinations of (i) the percentage of proliferation tumor cells; (ii) the PSA immunoreactivity score; and (iii) the proliferation/PSA ratio are shown in Table 15.

In one aspect of the invention, the tumor-derived material is obtained from a primary tumor. In connection with this aspect, the invention further comprises determining to the metastatic potential of a primary tumor, wherein high aggressiveness is associated with high metastatic potential and low aggressiveness is associated with low metastatic potential.

The term “metastatic potential” means the tendency of a primary tumor to form secondary metastatic lesions, resulting in the spread (metastasis) of the lesion from the primary site to a different or secondary site within the host's body. The newly pathological sites are referred to as “metastases”. A patient who is suffering from metastases has a lethal form of prostate cancer and a patient with high risk of developing metastases thus has a poor prognosis.

The method as defined above comprises the following preferred features:

(I) A PSA immunoreactivity score of 9 or lower, preferably 8 or lower, more preferably 6 or lower, is associated with high tumor aggressiveness.

(II) A fraction of 3% or more, preferably 4% or more, more preferably 5% or more, even more preferably 6% or more, most preferably 5.4% or more, proliferating cells is associated with high tumor aggressiveness.

(III) A fraction of 5.4% or more proliferating cells in combination with a PSA immunoreactivity score 9 or lower is associated with high tumor aggressiveness.

(IV) A proliferation-PSA combination score corresponding to a proliferation/PSA ratio of 0.2 or higher, more preferably 0.3 or higher, even more preferably 0.4 or higher, yet more preferably 0.5 or higher, yet more preferably 0.6 or higher, most preferably 0.7 or higher, is associated with high tumor aggressiveness.

In a further aspect, the invention provides a method for determining the need of curative prostate cancer treatment in a subject, said method comprising using the method as defined above for determining tumor aggressiveness in a subject diagnosed with prostate cancer, wherein

(i) a high tumor aggressiveness indicates the need for curative prostate cancer treatment in the subject; and

(ii) a low tumor aggressiveness indicates that active surveillance for prostate cancer is a safe treatment option.

The term “active surveillance” means a treatment plan that involves closely watching a patient's condition but not giving any treatment unless there are changes in test results that show the condition is getting worse. Active surveillance may be used to avoid or delay the need for treatments such as radiation therapy or surgery, which can cause side effects or other problems. During active surveillance, certain exams and tests are done on a regular schedule. Related terms include “watchful waiting” and “expectant management”.

In a further aspect, the invention provides a method as defined above (wherein the tumor-derived material is obtained from a primary tumor) for determining tumor aggressiveness in a subject diagnosed with prostate cancer and having a metastasis.

Such a method comprises the following preferred features:

(I) A PSA immunoreactivity score of 9 or lower, preferably 8 or lower, more preferably 6 or lower, is associated with high tumor aggressiveness.

(II) A fraction of 6% or more, preferably 9% or more, more preferably 12% or more, yet more preferably 15% or more, most preferably 17% or more proliferating cells is associated with high tumor aggressiveness.

(II) A fraction of 16% or more proliferating cells in combination with a PSA immunoreactivity score 9 or lower is associated with high tumor aggressiveness.

(IV) A proliferation-PSA combination score corresponding to a proliferation/PSA ratio of 1.8 or higher, more preferably 2.0 or higher, even more preferably 2.5 or higher, yet more preferably 3.0 or higher, yet more preferably 3.3 or higher, most preferably 2.1 or higher, is associated with high tumor aggressiveness.

In a further aspect, the invention provides a method for predicting the likelihood of effectiveness of prostate cancer treatment comprising androgen deprivation therapy and/or androgen receptor targeting therapy, said method comprising using the method, as defined above, for determining tumor aggressiveness in a subject diagnosed with prostate cancer and having a metastasis, wherein

(i) a low tumor aggressiveness indicates that androgen deprivation therapy and/or androgen receptor targeting therapy alone is likely to be effective in the subject; and

(ii) a high tumor aggressiveness indicates that androgen deprivation therapy and/or androgen receptor targeting therapy alone is not likely to be effective in the subject and that additional therapy is warranted.

The term “androgen deprivation therapy” (ADT) means antihormone therapy aiming at treating prostate cancer. ADT reduces the levels of androgen hormones, with surgery or drugs (chemical castration), to prevent the prostate cancer cells from growing. Chemical castration includes treatment with GnRH/LHRH analogs or antagonists.

The term “androgen receptor targeting therapy” means therapy that include the use of androgen receptor antagonists, such as bicalutamide, enzalutamide, apalutamide, darolutamide, and others under development for treatment of prostate cancer, or steroidogenesis inhibitors such as abiraterone, ketoconazole, galeterone, and others under development for treatment of prostate cancer.

In a further aspect, the invention provides a method for determining the need for prostate cancer treatment comprising chemotherapy and/or therapy using DNA repair inhibitors, said method comprising using the method, as defined above, for determining tumor aggressiveness in a subject diagnosed with prostate cancer and having a metastasis, high tumor aggressiveness indicates the need for chemotherapy and/or therapy using DNA repair inhibitors in the subject.

The term “chemotherapy” (often abbreviated to chemo and sometimes CTX or CTx) means a type of cancer treatment that uses one or more anti-cancer drugs (chemotherapeutic agents). Chemotherapy may be given alone or with other treatments, such as surgery, radiation therapy, or biologic therapy.

Taxane chemotherapy, given with prednisone, is a standard treatment for men with metastatic prostate cancer that has spread and is progressing despite hormone therapy. Taxane chemotherapy agents approved for the treatment of advanced prostate cancer include docetaxel (Taxotere®) and cabazitaxel (Jevtana®).

Platinum-based chemotherapy agents including carboplatin (Paraplatin®), cisplatin (Platinol®), and oxaliplatin (Eloxatin®), are known for the treatment of various cancer types, including prostate cancer.

The term “DNA repair inhibitors” means PARP inhibitors and other DNA repair inhibitors under development for treatment of prostate cancer.

In yet another aspect, the invention provides a method of treating prostate cancer in a subject in need thereof, said method comprising:

(a) using the method as defined above for determining tumor aggressiveness in a subject diagnosed with prostate cancer and having a metastasis, and

(b) administering a prostate cancer treatment to the subject; wherein
(i) if the tumor aggressiveness is low, the subject is administered androgen deprivation therapy and/or androgen receptor targeting therapy; and
(ii) if the tumor aggressiveness high, the subject is administered (I) androgen deprivation therapy and/or androgen receptor targeting therapy, in combination with (II) chemotherapy and/or therapy using DNA repair inhibitors.

In a further aspect, the invention provides a method, as defined above, for determining tumor aggressiveness in a subject diagnosed with prostate cancer and having a tumor, wherein the said tumor-derived material is obtained from a metastasis, such as a bone metastasis. Such a method comprises the following preferred features:

(I) A PSA immunoreactivity score of 9 or lower, preferably 8 or lower, more preferably 7 or lower, even more preferably 6 or lower, most preferably 5 or lower is associated with high tumor aggressiveness.

(II) A fraction of 20% or more, preferably 25% or more, more preferably 30% or more proliferating cells in the sample is associated with high tumor aggressiveness.

(III) A fraction of 25% or more proliferating cells in combination with a PSA immunoreactivity score 8 or lower is associated with high tumor aggressiveness.

(IV) A proliferation-PSA combination score corresponding to a proliferation/PSA ratio of 2.1 or higher, more preferably 2.2 or higher, even more preferably 2.4 or higher, yet more preferably 2.6 or higher, most preferably 2.9 or higher is associated with high tumor aggressiveness.

In yet another aspect, the invention provides a method for predicting the likelihood of effectiveness of prostate cancer treatment comprising androgen deprivation therapy and/or androgen receptor targeting therapy, said method comprising using the method, as defined above, for determining tumor aggressiveness in a subject diagnosed with prostate cancer and having a metastasis, wherein the said tumor-derived material is obtained from a metastasis, wherein:

(i) a low tumor aggressiveness indicates that androgen deprivation therapy and/or androgen receptor targeting therapy alone is likely to be effective in the subject; and

(ii) a high tumor aggressiveness indicates that androgen deprivation therapy and/or androgen receptor targeting therapy alone is not likely to be effective in the subject, and that additional therapy is warranted.

The invention further provides a method for determining the need for prostate cancer treatment comprising chemotherapy and/or therapy using DNA repair inhibitors, said method comprising using the method, as defined above, for determining tumor aggressiveness in a subject diagnosed with prostate cancer and having a metastasis, wherein the said tumor-derived material is obtained from a metastasis, wherein a high tumor aggressiveness indicates the need for chemotherapy and/or therapy using DNA repair inhibitors in the subject.

The invention further provides a method of treating prostate cancer in a subject in need thereof, said method comprising:

(a) using the method, as defined above, for determining tumor aggressiveness in a subject diagnosed with prostate cancer and having a metastasis, wherein the said tumor-derived material is obtained from a metastasis, and

(b) administering a prostate cancer treatment to the subject; wherein
(i) if the tumor aggressiveness is low, the subject is administered androgen deprivation therapy and/or androgen receptor targeting therapy; and
(ii) if the tumor aggressiveness is high, the subject is administered (I) androgen deprivation therapy and/or androgen receptor targeting therapy, in combination with (II) chemotherapy and/or therapy using DNA repair inhibitors.

In order to be diagnostically useful, the levels/scores of the biomarkers discussed herein must be compared to a reference value (also known as a “cut-off”). Suitably, the reference value is obtained by determining the levels of the same markers (most preferably using similar methods and similar samples) from a control subject, or more preferably by obtaining an average value from a group of control subjects.

The skilled person will appreciate that the level of difference from the reference value that is taken as indicative of presence of a disorder will vary from case to case. Requiring larger difference will increase the specificity of the diagnostic method but sacrifices sensitivity; requiring smaller difference will increase sensitivity at the cost of decreased specificity.

The desirable levels of specificity and sensitivity will vary depending on the setting: for example, in some cases a very high specificity is necessary to avoid large numbers of false positives; in other cases, a high sensitivity may be prioritized instead and lower specificity accepted. The determined level of the biomarker is also likely to vary depending on characteristics of the particular analytical method used to assay the concentrations as well as the type of sample and handling of the sample. All these considerations are well known to the skilled person. Likewise, solutions to the issues presented above (e.g. determining the cut-off values) are within the reach of the skilled person by combining the teachings herein with mere routine experimentation and optimization.

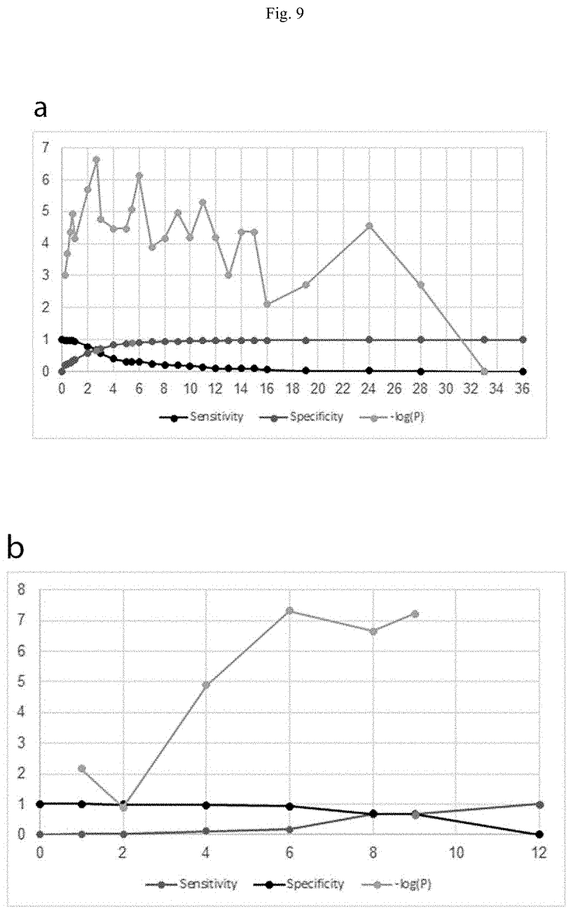

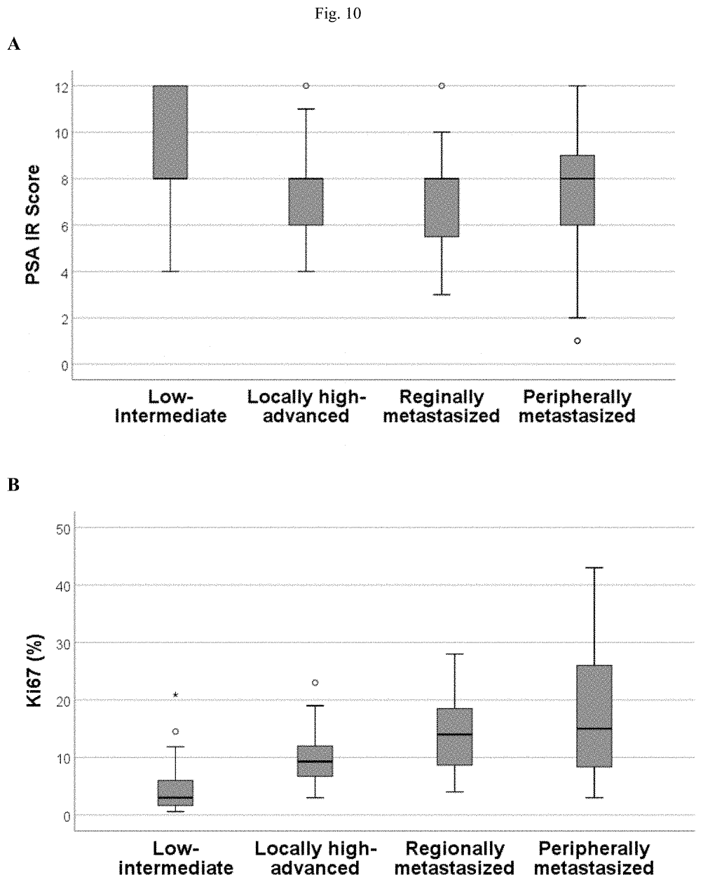

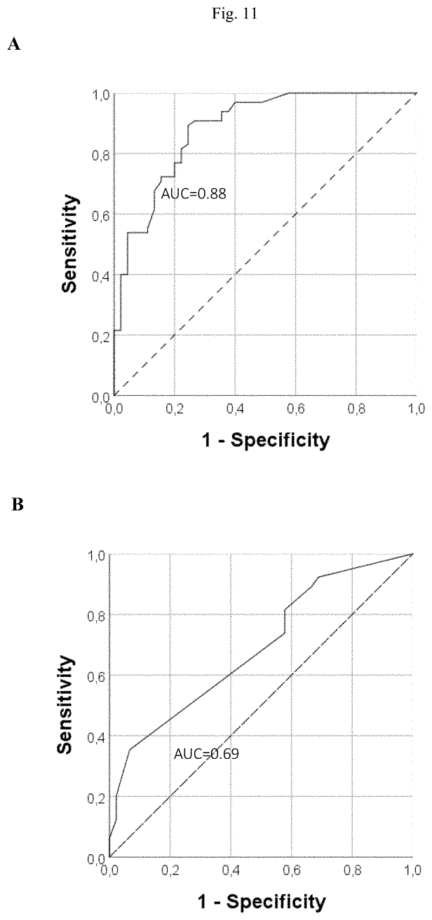

A statistical tool useful in determining the cut-off values is known as Receiver Operating Characteristic (ROC) curve, which may be constructed as follows. Rank all subjects (patients plus controls) after the measured parameter. Start from the upper part of the table and calculate, successively for each new measured value, the sensitivity and 100-specificity for all subjects (sensitivity=posP/allP, where posP is the number patients (patients meaning the subjects with the disease) that would be classified as having the disease using this measured value and specificity is negC/allC, where negC is the controls that are not classified having the disease). Plot these values in an x-y-diagram where “100-specificity is x” and sensitivity is y, resulting in a ROC-curve. The cut-offs are always adjusted to the actual situation including prevalence of the disease and especially the degree of severity of the disease but statistical programs (knowing nothing about the clinical situation) usually calculate the cut-offs by minimizing the distance from the upper left corner of the ROC-curve, i.e. minimizing ((100-sensitivity)\hat{ }2+(100-specificity)\hat{ }2), where \hat{ } means squared (Pythagoras theorem). The values of sensitivity and selectivity shown in FIGS. 11, 12, 14(A), 15 and 16(A) were obtained using this method.

In further aspects, the invention comprises the following numbered embodiments as disclosed in Swedish patent application No. 1950232-7, from which priority is claimed:
1. A method for determining tumor aggressiveness in a subject diagnosed with prostate cancer and having a tumor, said method comprising:
   (a) obtaining a sample comprising tumor-derived material from the said subject;
   (b) evaluating the level of cell differentiation in the sample; and
   (c) evaluating the level of proliferating cells in the sample;
   wherein
   (i) a low level of cell differentiation and a high level of proliferating cells are associated with high tumor aggressiveness, and
   (ii) a high level of cell differentiation and a low level of proliferating cells are associated with low or moderate tumor aggressiveness.
2. The method according to embodiment 1 wherein the said tumor is a metastasis.
3. The method according to embodiment 2 wherein the said tumor is a bone metastasis.
4. The method according to embodiment 1, comprising determining the metastatic potential of a primary tumor.
5. The method according to any one of embodiments 1 to 4 wherein evaluating the level of cell differentiation comprises evaluating the level of prostate-specific antigen (PSA).
6. The method according to embodiment 5 wherein evaluating the level of PSA in the sample comprises evaluating PSA immunoreactivity.
7. The method according to embodiment 6 wherein when the tumor is a primary tumor a PSA immunoreactivity score of 9 or lower is associated with high tumor aggressiveness; and when the tumor is a metastasis a PSA immunoreactivity score of 6 or lower is associated with high tumor aggressiveness.
8. The method according to any one of embodiments 1 to 7 wherein evaluating the level of proliferating cells in the sample comprises evaluating the level of Ki67.
9. The method according to embodiment 8 wherein evaluating the level of Ki67 in the sample comprises evaluating Ki67 immunoreactivity.
10. The method according to any one of embodiments 1 to 9 wherein when the tumor is a primary tumor a fraction of 5.4% or more proliferating cells is associated with high tumor aggressiveness; and when the tumor is a metastasis a fraction of 25% or more proliferating cells in the sample is associated with high tumor aggressiveness.

### EXPERIMENTAL METHODS

Patient Samples:

Samples of bone metastases were obtained from a series of fresh-frozen and formalin-fixed paraffin embedded (FFPE) biopsies collected from prostate cancer patients (n=72) operated for metastatic spinal cord compression at Umeå University Hospital (2003-2013). Primary tumor biopsies (FFPE) were available in in 52 cases. The patient series and the tissue handling have been previously described (4, 7, 10). Patients gave their informed consent and the study was conducted in accordance with the Declaration of Helsinki.

Primary tumor samples were also obtained from a historical cohort of 419 men with prostate cancer, detected after transurethral resection of the prostate (TURP) due to voiding symptoms, 1975-1991, in Vasteras, Sweden, for details see (11, 12). Patients with symptomatic metastases were treated with ADT, a few patients were treated with radiation or radical prostatectomy, while a majority of men were followed with expectancy (“watchful waiting”) according to clinical practice at that. All cases were Gleason regraded by a single pathologist.

For validation studies, a set of primary tumor biopsies were used, obtained from prostate cancer patients consecutively treated at the Umeå University Hospital as part of the Uppsala Umeå Cancer Consortium (UCAN) between 2013 and 2015. Patients to were categorized into different risk groups based on the following characteristics; low to intermediate risk (T1-2, GS≤7 and PSA<20 ng/ml, n=45), locally high to advanced (GS≥8 or T3 or PSA≥20 but below 50 ng/ml, n=21), regionally metastasized (T4 or N1 or PSA≥50 but below 100 ng/ml, n=16), and peripherally metastasized (M1 or PSA≥100 ng/ml, n=28). Patients treated with ADT due to metastasized disease were monitored for time to disease progression (TTP), defined as clinical or biochemical progress.

RNA Extraction and Gene Expression Analysis:

RNA was extracted from representative areas of fresh frozen bone metastases sections using the Trizol (Invitrogen, Carlsbad, Calif.) or the AllPrep DNA/RNA/Protein Mini Kit (QIAGEN, Hilden, Germany) protocols. Nucleic acids were quantified by absorbance measurements using a spectrophotometer (ND-1000 spectrophotometer; NanoDrop Technologies Inc, Wilmington, Del.). The RNA quality was analyzed with the 2100 Bioanalyzer (Agilent Technologies, Santa Clara, Calif.) and verified to have an RNA integrity number ≥6. Whole genome expression array analysis was performed using the human HT12 Illumina Beadchip technique (Illumina, San Diego, Calif.) with version 3 in (4) and version 4 in (7).

Bead chip data from two separate gene expression studies (GEO Datasets GSE29650 and GSE101607) were combined for all probes with average signals above twice the mean background level in at least one sample per study array. Arrays were individually normalized to remove batch effects, using the quantile method followed by centering of the data by subtracting the mean signal for each probe. Normalized datasets were merged by mapping Illumina ID and Hugo gene symbol. Redundant transcript probes were removed by selecting the probe with the highest median expression, leaving 10784 gene transcripts for subsequent analysis. When merging bead chip data with external RNA sequencing data (50) in class discriminant analysis (below), data was centered by dividing intensities for each gene product by the median in each cohort.

Multivariate Data Analysis:

Principal component analysis (PCA) was used to get an overview of the variability in data and to detect potential subgroups by unsupervised pattern recognition. Sevenfold cross-validation testing was used to assess the reliability of the model. Cluster analysis was performed based on the first m (m=2, 5) principal components, using five clustering algorithms: i) Hierarchical clustering using the Euclidian distance and Ward linkage, ii) Hierarchical clustering using the Manhattan distance and Ward linkage, iii) k-means clustering, iv) Self Organizing maps and v) Affinity propagation (13).

A prediction model for subtype was built using orthogonal projections to latent structures discriminant analysis, OPLS-DA (51), based on levels for the top 60 gene products differentiating one sample cluster from the others (defined by the lowest P values in Mann-Whitney U test and a median fold change ≥1.5), and applied to an external cohort of 43 bone metastases (50). OPLS-DA maximizes the explained variation in data (X) and its covariation with class membership, Y, defined by a dummy matrix of zeros and ones. Class membership was defined as software default, by predicted value i) <0.35 do not belong to the class, ii) between 0.35 and 0.65 intermediate and iii) above 0.65 belong to the class. Multivariate data modelling was performed with SIMCA software version 15.0 (Umetrics AB, Umeå, Sweden).

Functional Enrichment Analysis:

Gene set enrichment analysis (GSEA) was performed by the MetaCore software (GeneGo, Thomson Reuters, New York, N.Y.). Analysis was based on gene transcripts significantly increased in one cluster compared to the others, as defined by Kruskal Wallis followed by Mann-Whitney U test and adjusted P values (False Discovery Rate, FDR, <0.01). Sets of genes associated with a functional process (pathway map or network) were determined as significantly enriched per subtype based on P values representing the probability for a process to arise by chance, considering the numbers of enriched gene products in the data vs. number of genes in the process. P values were adjusted by considering the rank of the process, given the total number of processes in the MetaCore ontology. Possible drivers of each subtype were identified by exploring the relations between subtype-enriched transcripts and upstream regulators defined from the literature. P-values were calculated for connectivity ratios between actual and expected interactions with objects in the data.

Metastases and Primary Tumor Morphology:

The fraction of tumor epithelial cells in metastasis tissue was determined using stereological techniques, as earlier described (14). Metastasis cell atypia was graded either as moderate or pronounced and glandular differentiation was scored as observed or not. Cancer cells in metastases and primary tumor biopsies were stained and scored for AR, PSA, Ki67, and chromogranin-A as earlier described (10).

The PSA staining, using the A0562 PSA antibody (Dako) were quantified using a scoring system based on the percentage (0=no staining, 1=1-25%, 2=26-50%, 3=51-75% and 4=76-100% of tumor epithelial cells stained) and intensity (0=no staining 1=week, 2=moderate and 3=intense) of immunostained tumor epithelial cells. An immunoreactivity (IR) score was obtained by multiplying the scores for distribution and intensity, as earlier described (10), giving IR scores in the range of 0-12. Ki67 staining, using the anti-Ki-67 (30-9) Rabbit Monoclonal Primary Antibody (Roche Diagnostics), was quantified as the percentage of stained tumor epithelial cells (10). Combinatory PSA and Ki67 immunoreactivity scores were obtained using cut-offs at median or the upper quartile (per sample cohort), and by this patients were categorized into 4 different groups 1) PSA high/Ki67 low, 2) PSA high/Ki67 high, 3) PSA low/Ki67 low, and 4) PSA low/Ki67 high.

The stroma in primary tumor biopsies was scored for the percentage of AR positive cells as earlier described (15) and for a reactive desmoplastic response, characterized by loss of stroma smooth muscle and increase in fibroblasts and collagen, using a 3-tier scoring system (16).

Univariate Statistics and Survival Analysis:

Continuous variables were given as median (25th; 75th percentiles) and non-parametric statistics was used (Mann-Whitney U test, Wilcoxon test, Spearman rank correlation). The Chi-square test was used for categorical values. Survival analysis was performed by Kaplan-Meier analysis with death of prostate cancer as event and death by other causes as censored events and with follow-up time considering time from diagnosis or time from first ADT until the latest follow-up examination. The log-rank test was used to test for statistical significance in differences in survival. Cox proportional hazard models were used and results presented as hazard ratio (HR) with 95% confidence intervals. All tests were two sided and P-value less than 0.05 were considered statistically significant. Statistical analyses were performed using the Statistical Package for the Social Sciences, SPSS 24.0 software (SPSS, Inc, Chicago, USA). ROC analysis of patients treated with watchful waiting was performed with prostate cancer death as event. ROC analysis was also performed with the purpose of separating low-intermediate tumors from more aggressive tumors.

### EXAMPLES OF THE INVENTION

### Example 1: Global Gene-Expression in Bone Metastases and Identification of Robust Molecular Subtypes

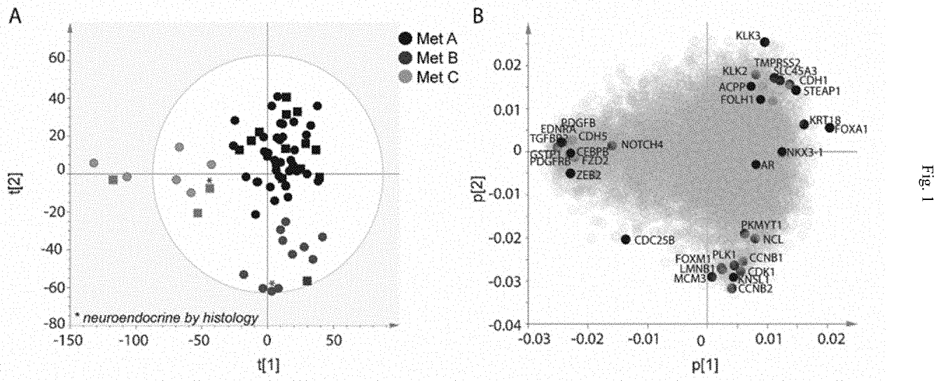

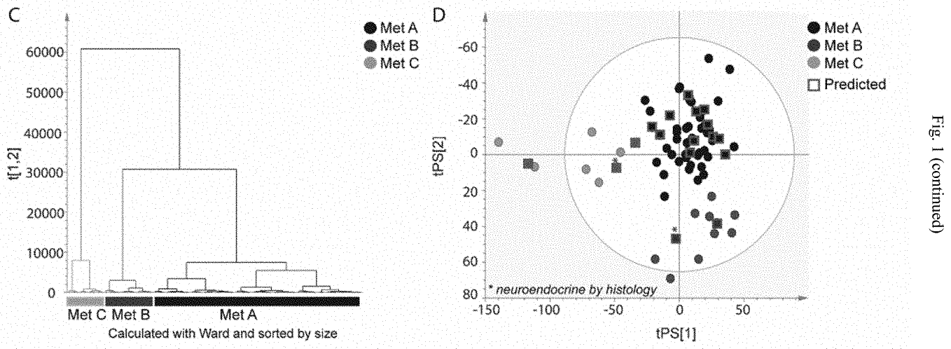

The global gene-expression pattern in 12 treatment-naive, 4 short-term castrated, and 56 CRPC bone metastases was explored. Based on transcript levels of 10784 non-redundant genes, a principal component analysis (PCA) model was built that included 9 significant principal components explaining 40% of the variation in the data. Hierarchical cluster analysis using the Euclidian distance and the first two principal components revealed three molecular subtypes of bone metastasis, referred to as metastasis subtype A, B, and C (MetA-C) (FIG. 1). The majority of samples clustered as MetA (71%), while 17% and 12% clustered as MetB and MetC, respectively (FIG. 1a-c), based on the loadings (gene expression levels) in FIG. 1c.

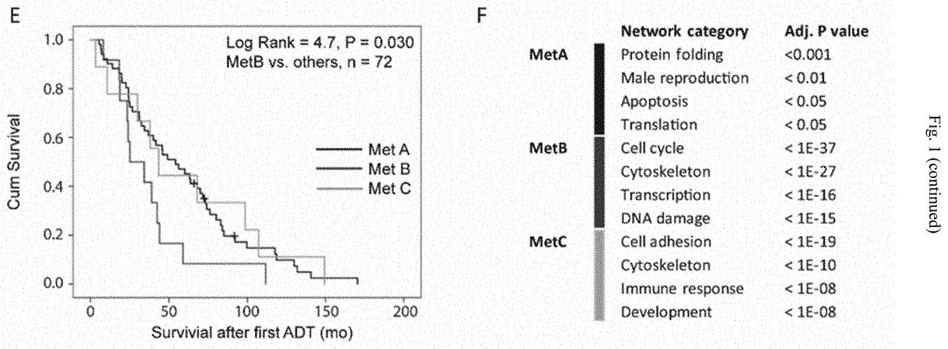

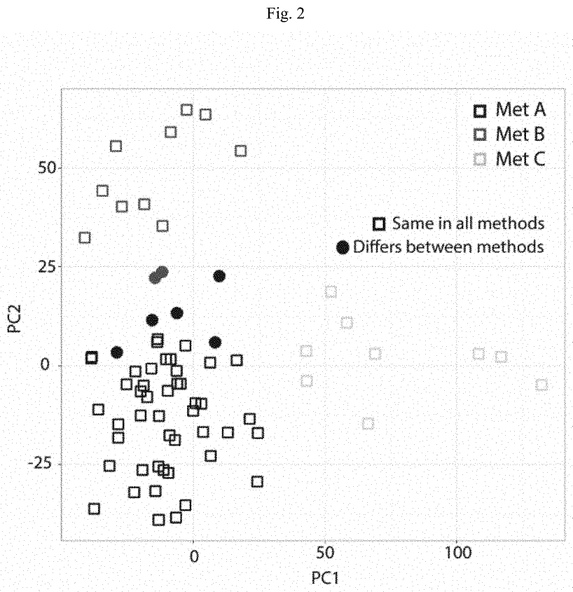

The inclusion of 5 principal components and the use of alternative clustering methods verified robust clustering with preserved grouping of 90% of the samples, and 90%, 83% and 100% consistency for the MetA, MetB and MetC samples, respectively (FIG. 2). Importantly, the MetA-C clusters were identified also when data analysis was based on CRPC samples only (FIG. 1d), leaving samples from treatment-naive and short-term castrated patients outside the PCA modelling together with two CRPC samples defined as neuroendocrine (NE, based on high chromogranin A and low PSA, AR expression). Those samples were predicted with 100% consistency and previously untreated metastases were identified within all clusters (FIGS. 1a and 1d), indicating that the MetA-C subtypes are intrinsic and not developed by the introduction of castration therapy.

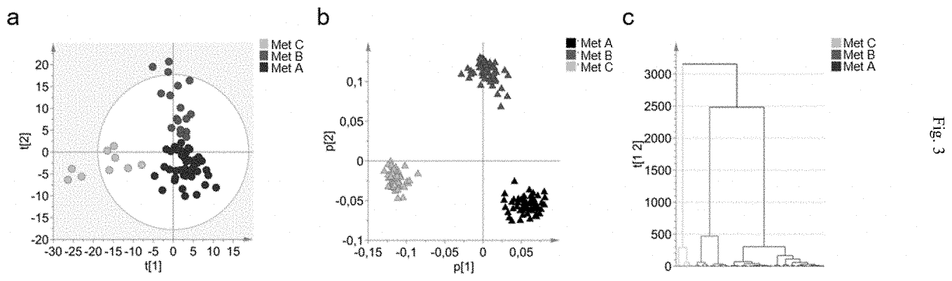

To enable validation of the MetA-C subtypes in an external data set of prostate cancer bone metastases (50), the top 60 gene products differentiating each sample cluster from the others (Table 1) were identified and used for PCA and OPLS-DA modelling (FIG. 3). Expression levels for the MetA-, MetB-, and MetC-associated genes, respectively, were highly correlated also within the external cohort and responsible for differentiating samples into three clusters (FIG. 3a-f). Accordingly, the MetA-C subtypes in the validation cohort were predicted at frequencies comparable to those originally observed (FIG. 3g-i).

### Example 2: Metastasis Subtypes Relate to Patient Characteristics and Prognosis

As can be seen in Table 2, most patients were diagnosed with locally advanced or metastatic disease; high serum PSA levels, and poor tumor differentiation (high Gleason score, GS). In patients where prostate cancer was not diagnosed until it caused neurological symptoms (patients without ADT at metastasis surgery), the primary tumor was not biopsied. Most patients were directly treated with ADT, while 10 patients had been previously treated with curative intent (Table 2). In 52 cases (72%) there were available primary tumor biopsies for morphological analysis. At relapse to castration resistance, patients had been given second line treatments as indicated (Table 2).

To assess the clinical relevance of the molecular subtypes, MetA-C were analyzed in relation to the patient characteristics summarized in Table 2. Patients with the MetB subtype had shorter cancer-specific survival after ADT than MetA and MetC patients (median survival 25 months vs. 49 months, respectively, P=0.030, FIG. 1e), and lower serum PSA levels compared to MetA patients at diagnosis (0.28-fold, P=0.011) and borderline at metastasis surgery (Table 2). A tendency of low PSA levels was seen also in MetC patients (Table 2). As described above, the subtypes were not related to previous ADT (FIG. 1), while a relatively high proportion of MetB patients had undergone radiation therapy to primary tumor (P=0.006) and received bicalutamide and/or chemotherapy subsequent to ADT (P=0.038 and 0.017, respectively, Table 2). This discrepancy in treatment history may be related to the particularly aggressive clinical course and poor response to ADT in MetB patients (FIG. 1e). Neither primary tumor Gleason score (GS) nor patient age or soft tissue metastasis were significantly associated with any specific subtype.

### Example 3: Metastasis Subtypes have Different Morphology

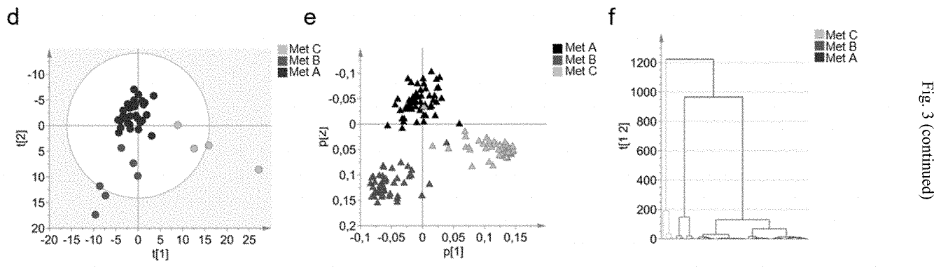

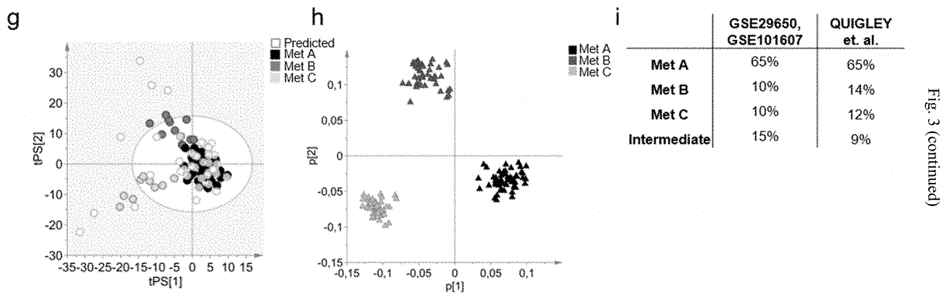

Most metastases were poorly differentiated with sheets of tumor epithelial cells resembling Gleason grade 5. while some showed patterns similar to Gleason grade 4 (FIG. 4a-c). Some metastases showed a prominent connective tissue stroma (FIG. 4a-c). The fraction of cancer cells was significantly lower in MetC compared to MetA tumor sections (Table 2) Importantly. this was seen both in the frozen sections (used for gene-expression analysis) and in the paraffin-embedded tissue (used for morphology analysis) representing distinct metastasis areas from the same patient, suggesting intrinsic differences in epithelium/stroma ratio between subtypes. Additional subtype-related differences were identified based on histological and immunohistochemical analysis of markers previously associated with aggressive prostate cancer (summarized in Table 3). with the most pronounced being reduced tissue PSA. increased proliferation (fraction of Ki67-stained tumor cells), cellular atypia and lack of glandular structures in MetB. Marked intra-tumor heterogeneity in immune-staining pattern was observed, as previously reported (10).

### Example 4: Enrichment of Divergent Functional Processes Per Metastasis Subtype

To identify subtype-enriched functional processes, gene transcripts with significantly increased levels per subtype were subjected to GSEA in the MetaCore software. Network analysis showed enrichment of protein translation and folding, male reproduction and regulation of apoptosis in MetA; cell cycle and DNA damage response, cytoskeleton reorganization and transcription in MetB; and cell adhesion, cytoskeleton, immune response, and development in MetC (FIG. 1f).

Pathway analysis demonstrated enrichment of “AR activation and downstream signaling in prostate cancer” in MetA compared to other subtypes, based on high transcript levels of KLK3 and other canonically AR-regulated genes such as KLK2, FOLH1, STEAP1, TMPRSS2, SLC45A3, ACPP (PPRP), and CDH1 (FIG. 1b). MetA also showed high expression of the luminal cell marker KRT18 (FIG. 1b) and enrichment of metabolic pathways involving amino acid and fatty acid degradation.

The MetB subtype showed pathway enrichment representing all phases of the cell cycle, including “Initiation of mitosis”, based on high FOXM1, CCNB1, CCNB2, CDC25B, CDK1, PLK1, PKMYT1, LMNB1, KNSL1, and NCL expression (FIG. 1b,) Other markedly enriched pathways in MetB included response to DNA damage and transcription. MetB expression levels of KRT18 were similar to MetA, while most luminal cell markers like as KLK3 and CDH1 were reduced, indicating luminal cell dedifferentiation coupled to increased cell division.

Among many enriched pathways in MetC, “ECM remodeling”, “regulation of EMT”, and “immunological synapse formation” were among the most prominent. Enrichment of “the EMT pathway” in MetC was based on high levels of transcripts involved in Wnt, Notch, TGF-beta, and PDGF signaling (FIG. 1b,). MetC showed low expression of luminal cell markers, but was enriched for some transcripts indicating a basal cell phenotype; i.e. CEBPB and GSTP1. Other basal cell markers like p63 and CK5 were low in all cases. Expression levels of luminal cell markers AR and NKX3.1 did not significantly differ between subtypes.

### Example 5: Possible Drivers of Metastasis Subtypes

As the MetB subtype was associated with the worst clinical outcome, putative drivers of its key characteristics, i.e. luminal cell dedifferentiation and proliferation, were identified. Based on connectivity analysis of gene networks and upstream regulators, a set of interesting candidate drivers were identified, such as the FOXA1 transcription factor (HNF3alpha) in MetA and the FOXM1 transcription factor in MetB. While FOXA1 may interact with the AR in MetA to drive canonical AR signaling and luminal differentiation (FIG. 1b,), FOXM1 may drive proliferation in MetB (FIG. 1b) (17).

Several kinases with inhibiting drugs available in the clinic for treatment of other cancer types or in clinical trials were suggested as upstream regulators for specific subtypes, e.g. ErbB2 (MetA), AURORA A/B (MetB), and PDGF-R-beta (MetC), hypothetically indicating possibilities for subtype-related therapeutic options.

### Example 6: Immunohistochemistry to Determine Metastasis Subtype

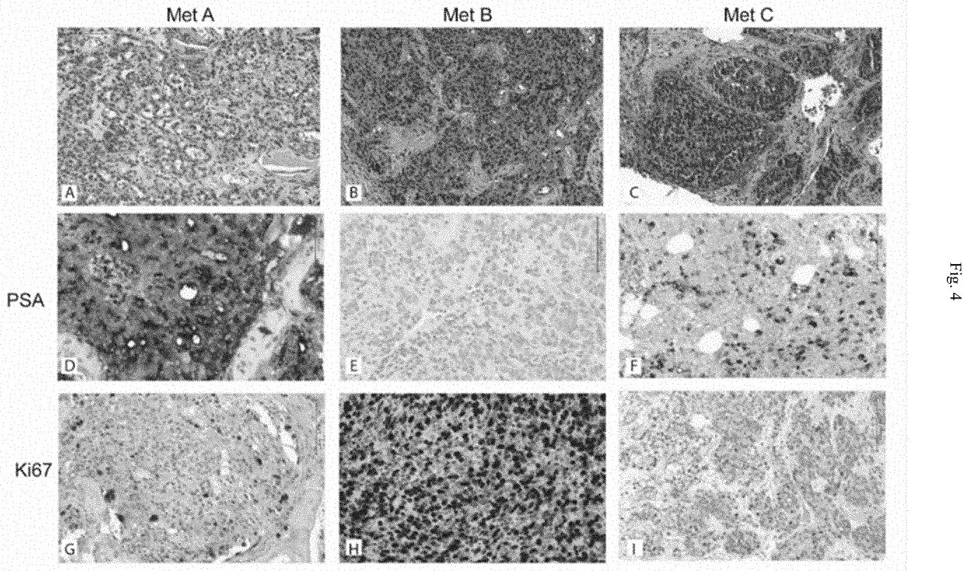

Based on gene expression data and morphological observations, PSA and Ki67 were selected as potential subtype-related surrogate markers (FIG. 4d-i, Table 3). Notably, the PSA staining score was higher in metastases with than without glandular differentiation (P=0.016, n=72) and in cases without pronounced atypia (P=0.012, n=72), suggesting that high cellular PSA is a marker for preserved epithelial and glandular differentiation in tumor cells. Accordingly, patients with low PSA staining scores (below median, scores 0-6) and high proliferation (fraction of Ki67 stained cells in the upper quartile, >25%), respectively, had short cancer-specific survival after first ADT in comparison to other patients (FIG. 5a-b). The PSA staining score inversely correlated to tumor cell proliferation in bone metastases (Rs=−0.32, P=0.007, n=71) (FIG. 5c), and a combinatory score identified 4 groups of metastases with the following frequencies; high PSA, low Ki67 (41%); low PSA, low Ki67 (32%); low PSA, high Ki67 (18%); high PSA, high Ki67 (8.5%) (FIG. 5d). MetB samples were enriched among the low PSA, high Ki67 samples (9/13, 69%) whereas MetC was not specifically enriched by these markers. Patients with high PSA, low Ki67 were enriched for MetA (86%) and showed the best prognosis (FIG. 5d).

### Example 7: Comparisons Between Bone Metastases and Paired Primary Tumors

It was investigated whether subtype-related difference in metastases could be traced back to the corresponding primary tumors, by exploring morphologic factors in diagnostic needle biopsies, as summarized in Table 3 and demonstrated in FIG. 4j-r. Collectively, these observations indicated that characteristics of MetB, such as high proliferation and low tissue PSA, may be detectable already in the primary tumor (Table 3). Primary tumors of MetB patients also showed low AR staining in the tumor stroma coupled to a reactive stroma response (Table 3).

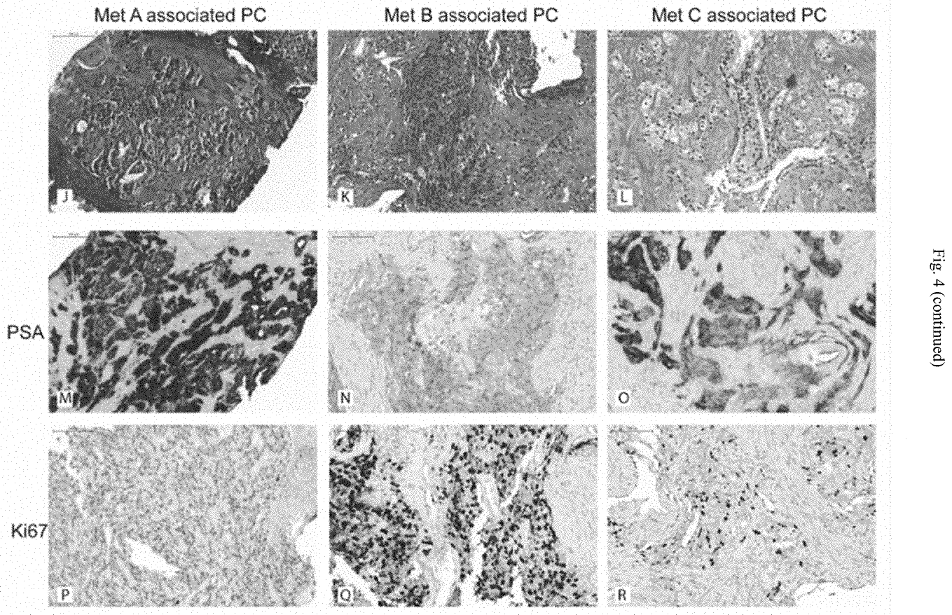

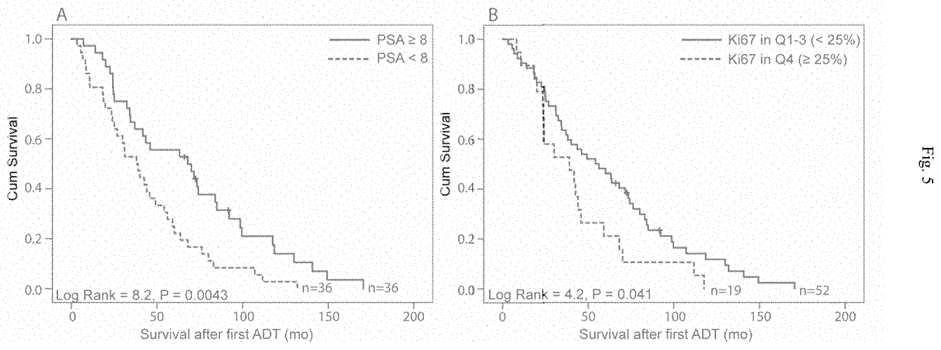

Paired-wise analysis showed significantly reduced AR (P=2.3E-5, n=34) and PSA (P=0.017, n=32) staining in MetA metastases compared to their corresponding primary tumors, while the fraction of Ki67 positive cells was significantly increased (P=0.013, n=35) (FIG. 6). Those markers did not significantly change from primary tumor to metastasis in MetB or MetC patients (FIG. 6).

### Example 8: Determining Prognosis by Analysis of Subtype-Related Markers in Primary Tumors

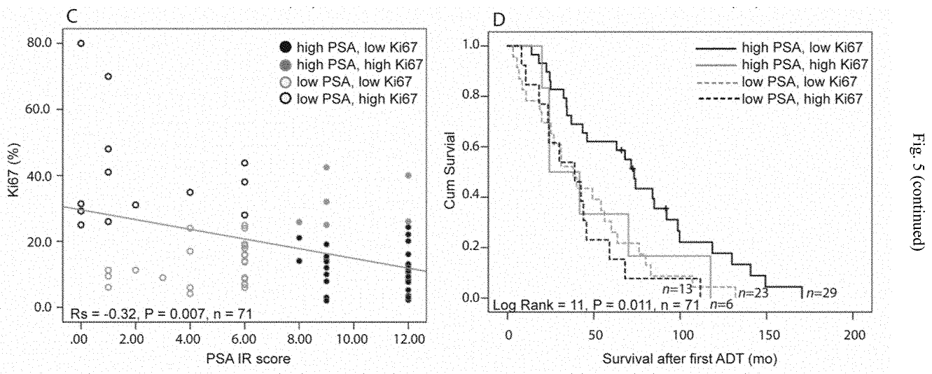

It was investigated whether surrogate immunohistochemical markers for the MetA and MetB phenotypes could differentiate patient outcome also if analyzed in primary tumor tissue. High Ki67 and low PSA immunoreactivity (MetB enriched) was associated with short survival after first ADT in two different cohorts; i) primary tumor biopsies of the MetA-C patients in the current study (FIG. 7a) and ii) TURP diagnosed cases (FIG. 7b), by using the PSA median and Ki67 upper quartile as cut-off values for the corresponding cohorts (PSA 8 and Ki67 16% in (i) and PSA 9 and Ki67 5.4% in (ii), respectively). Patients with the combination of high PSA and low Ki67 (MetA-enriched) had a more favorable outcome than other patients when treated by ADT (FIG. 7a-b). The combinatory PSA and Ki67 IR score provided independent prognostic information to GS in multivariate survival analysis (FIG. 7c-d).

### Example 9: Reduced Tissue PSA Level and Increased Ki67 Labelling are Related to Poor Outcome in Patients Treated with Watchful Waiting

Data were obtained from a historical cohort of men with prostate cancer detected after transurethral resection of the prostate (TURP) due to voiding symptoms. Immunohistochemical data for tumor cell proliferation (fraction of Ki67 positive cells) was available for 389 of the cases (11, 12). The available original tissue blocks were now sectioned and stained for PSA (n=347), as earlier described (10), resulting in combined Ki67 and PSA data in 332 cases. In non-malignant prostate tissue the glandular luminal cells showed intense PSA staining (score 3) in at least 75% of the glandular tissue (score 4), resulting in a PSA IR score of 12. This staining pattern was the most common also in prostate cancers, seen in 48% of the cases. However, in many men reduced PSA staining was seen in parts of or in the entire tumor, giving PSA IR score below 12.

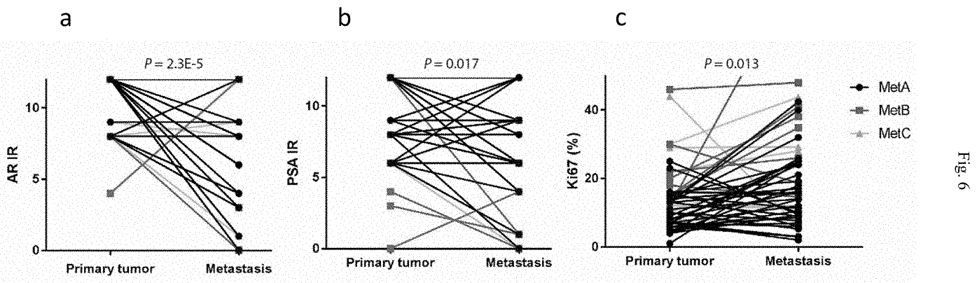

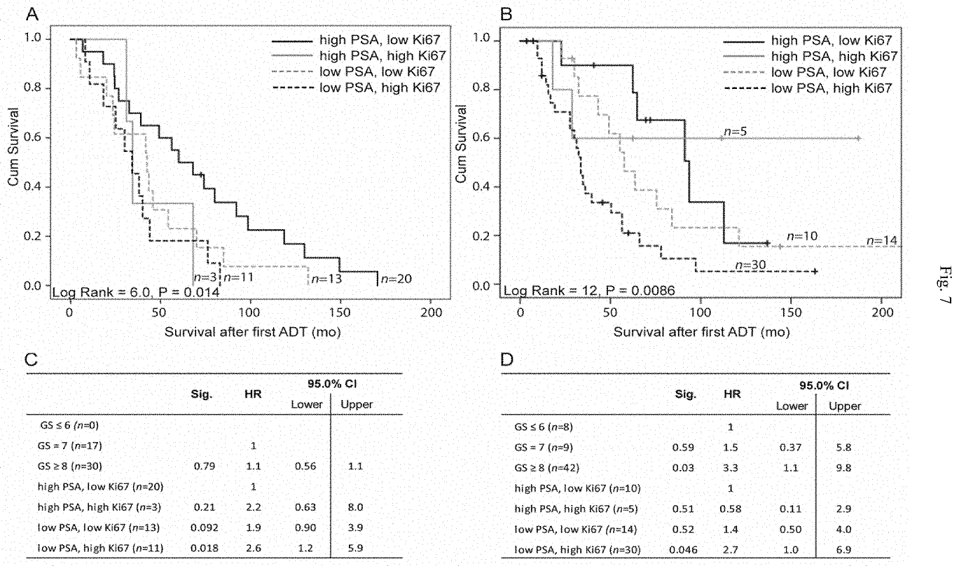

Men managed with watchful waiting and available PSA scores (n=247) were analyzed for cancer specific survival. Patients with a low PSA IR score (below 12) had short cancer specific survival compared to those with a PSA IR score of 12 (FIG. 8a). Specifically, low level of PSA staining was associated with poor prognosis also in men with GS≤6 (FIG. 8b).

In men managed with watchful waiting, increased Ki67 labeling above median and particularly in the highest quartile (Q4) were associated with a poor outcome as earlier described in more detail (11, 12).

### Example 10: Combined Analysis of PSA and Ki67 Immunoreactivity Identifies Patients with Different Prognosis when Treated with Watchful Waiting

The Ki67 and PSA immunostaining scores were moderately and inversely correlated (Spearman rank correlation=−0.46, p<0.001), but both variables provided independent prognostic information from GS in multivariate Cox survival analysis (Table 3). The PSA and Ki67 values were therefore used in combination. First, the median (med) IR scores; PSA (>9) and Ki67 (≥2.7%), were used as cut-off values for “high” levels and to separate tumors into 4 different groups:
(1) PSA high/Ki67 low;
(2) PSA high/Ki67 high;
(3) PSA low/Ki67 low; and
(4) PSA low/Ki67 high.

Kaplan-Meier survival analysis showed that these groups had different outcomes when managed by watchful waiting, with PSA high/Ki67 med-low being the most favorable and PSA low/Ki67 med-high the worst combination (FIG. 8c). This was true also for patients with GS≤6 (FIG. 8d). Patients with PSA high/Ki67 med-high and PSA low/Ki67 med-low cases showed intermediate prognosis.

In order to identify a subgroup of patients with a particularly poor prognosis, patients were divided into PSA/Ki67 groups using Q4 (≥5.4%) as the cut-off value for “Ki67 high”:
(1) PSA high/Ki67 Q4-low (121/237, 51%, of men managed by watchful waiting);
(2) PSA high/Ki67 Q4-high (11/237, 4.6%);
(3) PSA low/Ki67 Q4-low (78/237, 33%); and
(4) PSA low/Ki67 Q4-high (27/237, 11%).

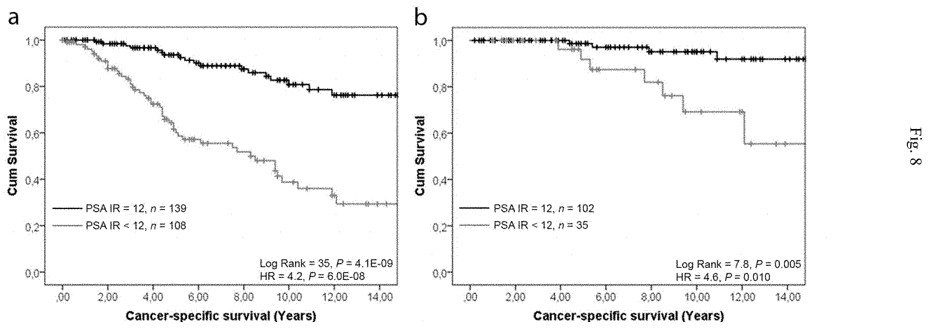

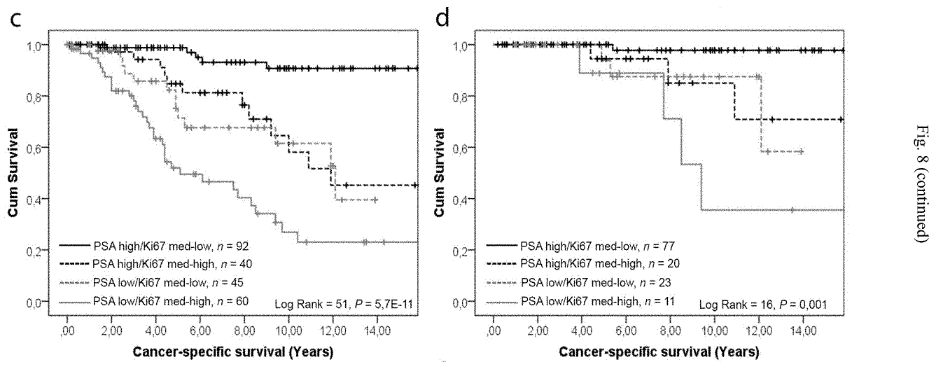

As anticipated, patients with PSA low/Ki67 Q4-high had the worst prognosis (FIG. 8e). Among the GS≤6 patients, PSA low/Ki67 Q4-high were very rare, but it was still obvious that reduced PSA and/or increased Ki67 levels were associated with poor prognosis (FIG. 80. Notably, the cut-off values for defining PSA/Ki67 high/low should be adjusted with the purpose of increasing sensitivity or specificity, respectively, in relation to the defined application (FIG. 9).

Taken together, those results indicated that a combinatory PSA and Ki67 IR score adds prognostic information to GS in prostate cancer patients (Table 4). Furthermore, for identification of patients with a good prognosis a lower Ki67 cut-off level seems superior whereas cases with a particularly poor prognosis are more specifically identified by increasing the Ki67 cut-off value.

### Example 11: Clinical and Histopathological Characteristics of Tumors Categorized by their PSA and Ki67 Immunoreactivity

As the identified subgroups based on PSA and Ki67 staining showed differences in clinical behavior, their characteristics were examined in more detail (using all available cases irrespective of treatment, and the Q4 was used to define high Ki67). The most common group, PSA high/Ki67 Q4-low (141/331, 43% of all cases), contained tumors with an IHC staining pattern similar to that of normal prostate to glands, that is homogeneous and intense PSA staining and low cell proliferation. This group was characterized by low GS, low tumor extent and stage, and low fraction of bone metastases at diagnosis (Table 5). Furthermore, they showed low values of various markers in the tumor epithelium and in the tumor stroma previously related to poor outcome in this patient cohort (Tables 5 and 6). Although the PSA high/Ki67 Q4-low subgroup showed the best prognosis, still 18% of the men in this group died from prostate cancer (see below). Using the median Ki67 as cut-off a smaller (106/331) PSA high/Ki67 med-low group where only 12% died from prostate cancer was identified.

The group most different from that above, defined by PSA low/Ki67 Q4-high (68/331, 21% of all cases) was characterized by high GS, high tumor volume and stage, many cases with bone metastases already at diagnosis, and in this group 74% of the patient died from prostate cancer (Tables 5 and 6, FIG. 8). Several markers previously associated with poor outcome showed levels suggesting particularly aggressive disease in this group (Tables 5 and 6). For example, the highest levels of pEGF-R, ErbB2, pAkt, and Erg as a marker for TRMPSS2-ERG fusion gene (38) were found in the tumor epithelium of this group. The tumor stroma showed signs of a reactive response (39, 40) with increased type 2 (CD163+) macrophage infiltration, vascular density and hyaluronic acid, and reduced levels of caveolin-1, androgen receptors and mast cells (Tables 5 and 6). All these tumor characteristics were seen also in the larger group (116/331) defined by PSA low/Ki67 med-high, a group where 66% of the men died from prostate cancer (data not shown).

The 2nd largest group (105/331, 32%) contained cases defined by PSA low/Ki67 Q4-low. Also this group had higher GS, tumor volume, stage, and fraction of cases with bone metastases at diagnosis than the PSA high/Ki67 Q4-low group (Table 5). They also had a less favorable outcome than the PSA high/Ki67 Q4-low group, but the prognosis was better than for the PSA low/Ki67 Q4-high group (Table 5, FIG. 8). Accordingly, markers previously found associated with a poor prognosis suggested that this group scored intermediate between the other groups. About 50% in this group died from prostate cancer (see below).

The group defined by PSA high/Ki67 Q4-high contained very few patients (17/331, 5%) suggesting that the phenotype is uncommon. This group of patients had higher tumor volume and stage and percentage of cases with bone metastases than the group with PSA high/Ki67 low, as well as significantly increased levels of ErbB2 and hyaluronic acid (Table 5).

It was investigated whether the tumor-instructed normal tissue (TINT) response (43) was associated with tumor subtype. Subgroups PSA high/Ki67 Q4-low and PSA low/Ki67 Q4-high, the groups with the best and worst prognosis, respectively, showed some morphological differences in the benign parts of the tumor bearing prostate. The benign parts of prostate carrying PSA low/Ki67 Q4-high tumors was characterized by significantly increased pEGF-R (P<0.01) in the epithelium and increased number of mast cells (P<0.01) in the stroma (Table 4). Epithelial pAkt (P=0.07) and Ki67 (P=0.07) in benign glands, and hyaluronic acid in the stroma (P=0.07) also tended to be increased.

As noted above, disease outcome differed within each subgroup. Patients dying from prostate cancer were compared to those that died from other causes or were alive. In the PSA high/Ki67 Q4-low tumors, the relatively few cases that died from prostate cancer had higher median GS (7 vs. 6, P<0.001), tumor stage; (2 vs. 1, P<0.05), tumor content (60 vs. 10%, P<0.001) and Ki67 index (2.7 vs. 1.2%, P<0.01). They also showed signs of a more pronounced stroma reaction with more hyaluronic acid (8 vs. 7, P<0.05), and blood vessels (14 vs. 11, P<0.05), as well as lower caveolin-1 in the tumor stroma (3 vs. 3, P<0.05) than those alive or dying from other causes. In the group with PSA low/Ki67 Q4-low where 51% died from prostate cancer, the men who died from prostate cancer had higher GS (8 vs. 6, P<0.001), higher tumor volume (75 vs. 30%, P<0.01), higher stage (3 vs. 1, P<0.001), and more commonly metastases at diagnosis (25 vs. 3%, P<0.01), but their PSA or Ki67 staining scores did not differ from those alive or dying from other causes. They also had higher hyaluronic acid staining in tumor stroma (9 vs. 7, P<0.01), more tumor infiltrating CD163+ macrophages (25 vs. 19, P<0.05), reduced stroma androgen receptors (42 vs. 52, P<0.05) and reduced caveolin-1 (2 vs. 3, P<0.05). The few patients dying from other causes in the PSA low/Ki67 Q4-high group had lower median GS (7 vs. 9, P<0.01) than those dying from prostate cancer. In summary standard prognostic markers like GS and the magnitude of stroma response affected prognosis within the PSA/Ki67 subgroups.

### Example 12: Reduced Tissue PSA Level and Increased Ki67 Labelling in Primary Tumor Biopsies are Related to More Aggressive/Progressive Prostate Cancer

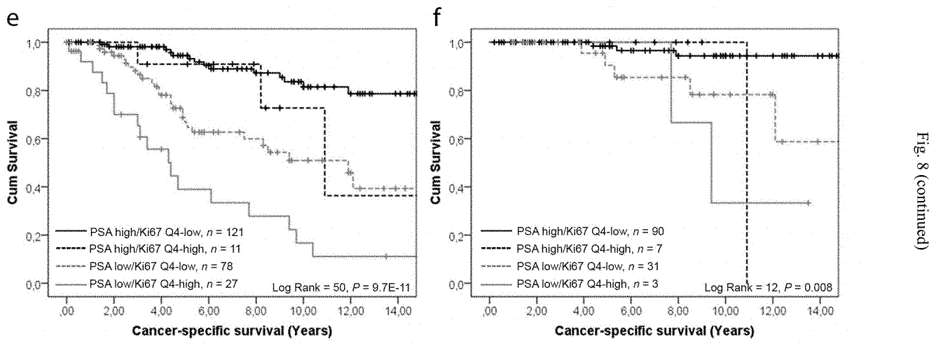

The PSA immunoreactivity score and fraction of Ki67 positive tumor cells were evaluated in primary tumor biopsies from patients with prostate cancer diagnosed within different risk groups, and were found to decrease respectively increase with disease progression (FIG. 10). ROC analysis was used to evaluate sensitivity and specificity of Ki67 and PSA immunoreactivity for differentiation of patients with indolent disease (low to intermediate risk tumors, n=45) from patients in need of therapy for progressive disease (high risk, locally advanced, regionally metastasized or peripherally metastasized, n=65), according to different cut-off levels (FIG. 11, Table 7-8). The results confirmed PSA=9 and Ki67=5-6 as suitable cut-off values for prognosticating progressive disease, although cut-offs could be adjusted to compensate for any requested sensitivity or specificity.

### Example 13: Reduced Tissue PSA Level and Increased Ki67 Labelling in Primary Tumor Biopsies Predicts Short Time to Progression after Androgen-Deprivation Therapy

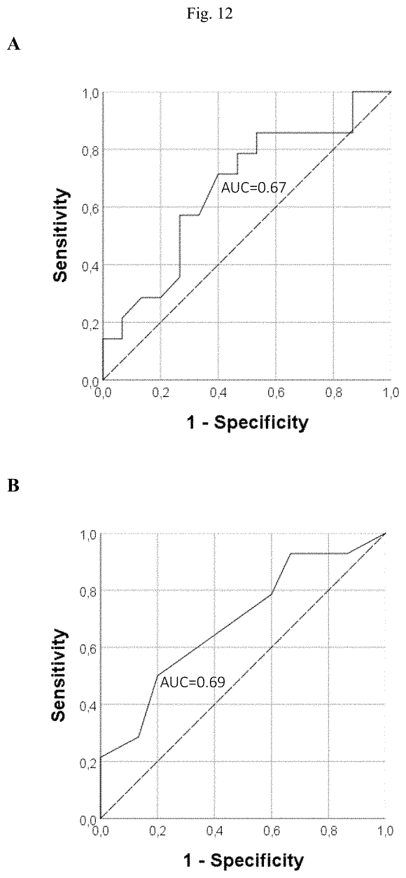

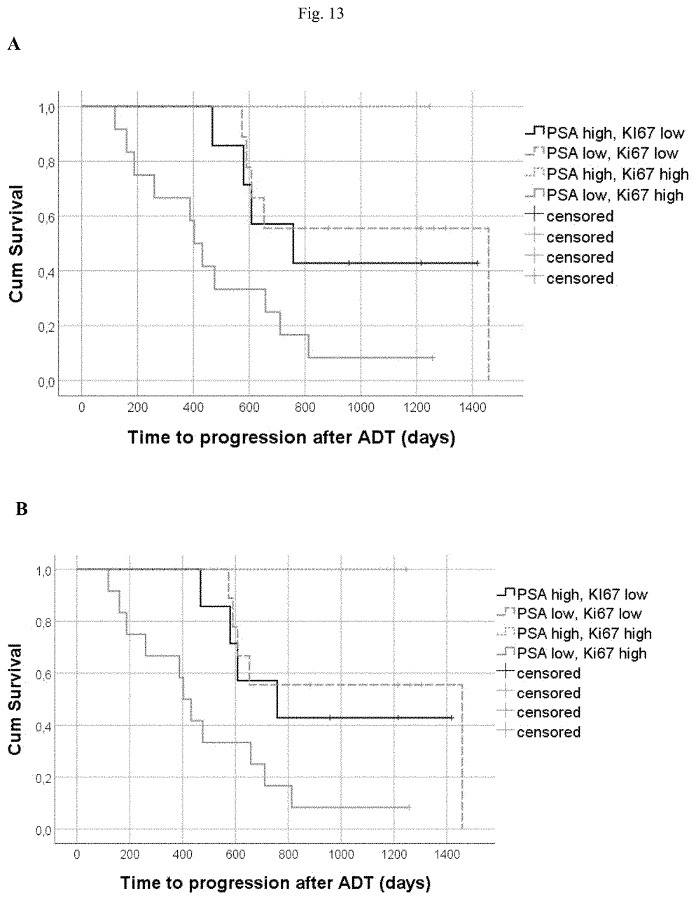

ROC analysis was used to evaluate sensitivity of Ki67 and PSA immunoreactivity in primary tumor biopsies for differentiation of patients with metastatic disease according to short or long time to progression after ADT, and to define suitable cut-off values (FIG. 12, Table 9-10). From this analysis, PSA≤8 and Ki67≥16 were chosen as optimal cut-off values and combined to divide patients into 4 groups; PSA high, Ki67 low; PSA high, Ki67 high; PSA low, Ki67 low, and PSA low, Ki67 high. Patients with low PSA and high Ki67 (PSA≤8 and Ki67≥16) were found to have particularly short TTP after ADT (FIG. 13A). When the same cut-offs values were also used to analyze cancer-specific survival in a TURP patient cohort receiving ADT due to symptoms from metastatic disease, patients with high PSA and low Ki67 (PSA>8 and Ki67>16) showed the best prognosis (FIG. 13B).

### Example 14. A High Ki67 to PSA Immunoreactivity Ratio is Related to Poor Prognosis and Predicts Short Time to Progression after Androgen-Deprivation Therapy

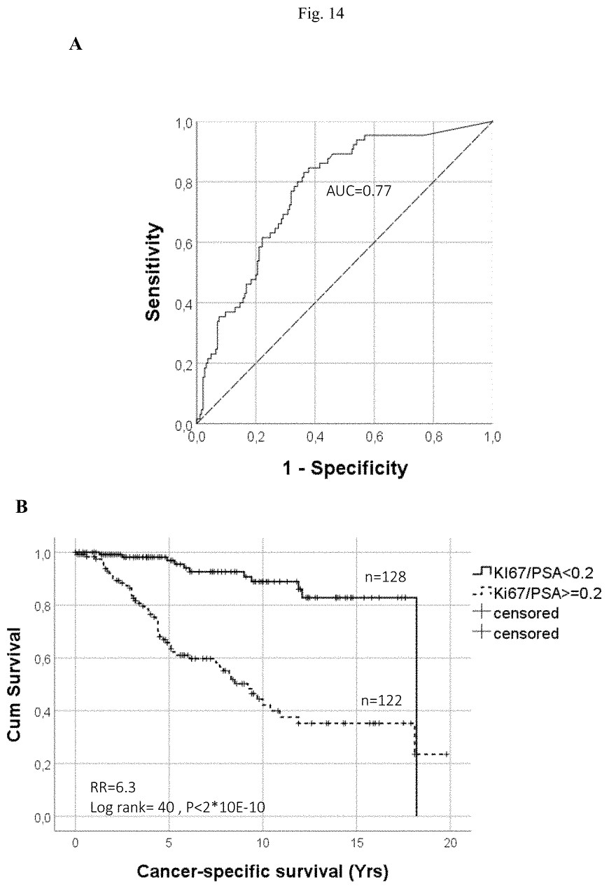

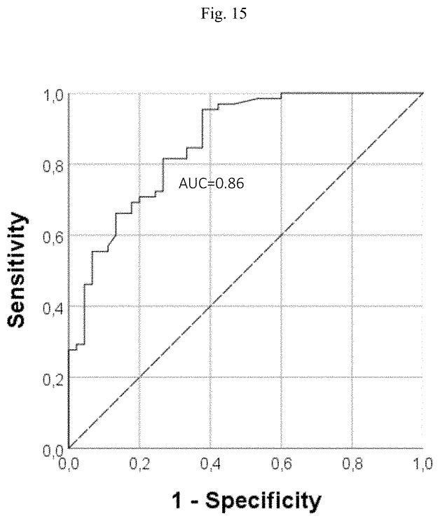

A Ki67/PSA-ratio was obtained for each tumor sample by dividing fraction of Ki67 positive tumor cells with the corresponding PSA immunoreactivity score. ROC analysis was applied to evaluate the sensitivity and specificity of the Ki67/PSA-score for identifying death from prostate cancer in patients diagnosed at TUR-P (1975-1991) and managed by watchful waiting (FIG. 14, Table 11). A Ki67/PSA-ratio of 0.2 was chosen as a suitable cut-off value for demonstrating increased risk of cancer-specific death in patients above compared to patients below cut-off (RR=6.3, P<2E-10, n=250, FIG. 15), although it is obvious from FIG. 14 and Table 11 that other cut-off values could be chosen in order to increase either test sensitivity or specificity. Multivariate Cox regression analysis confirmed that the Ki67/PSA-ratio added prognostic information to Gleason score (GS), related to cancer-specific survival in patients treated with watchful-waiting (Table 12).

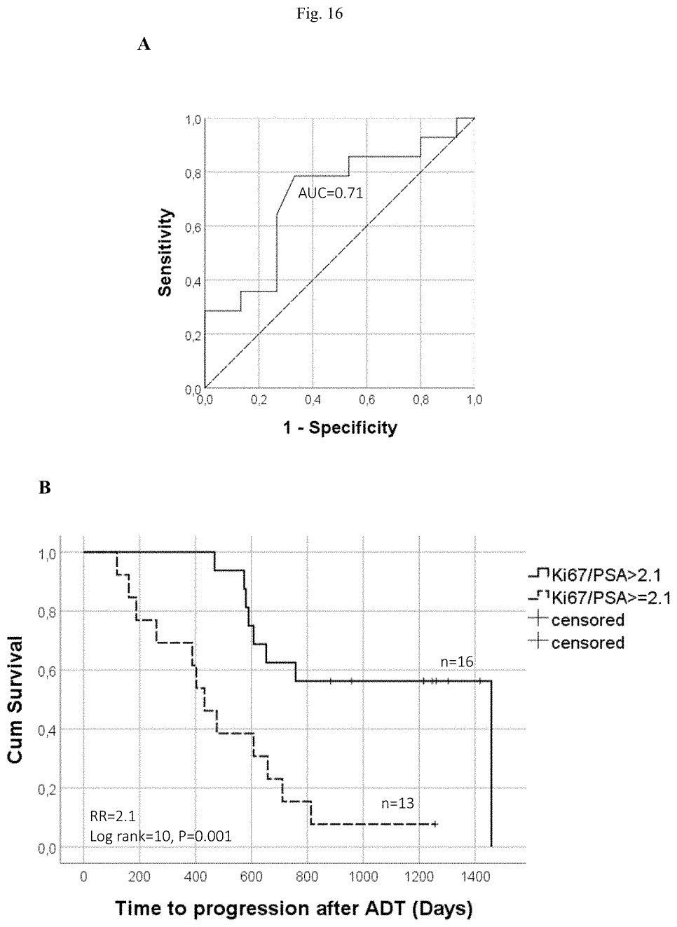

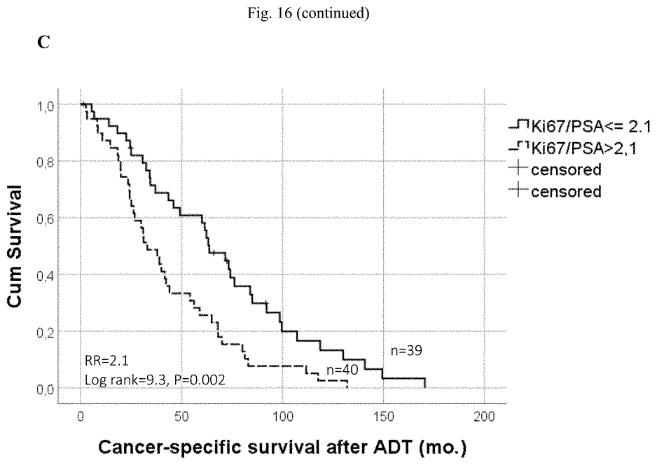

Accordingly, a Ki67/PSA-ratio of 0.2 were found to be a suitable cut-off value for differentiating patients with indolent disease (low to intermediate risk tumors, n=45) from patients in need of therapy for progressive disease (high risk, locally advanced, regionally metastasized or peripherally metastasized, n=65), when analyzing primary tumor biopsies within the UCAN cohort (FIG. 15, Table 13). When using the Ki67/PSA-ratio to differentiate patients with metastatic disease based on short or long time to progression after androgen-deprivation therapy (below or above median time), however, higher cut-off values needed to be applied (FIG. 16A, Table 14). As exemplified by choosing the cut-off value 2.1 from Table 14, patients with Ki67/PSA>2.1 in their primary tumor biopsies had a 2.1-time increased risk of short time to progression after ADT compared to patients with a lower Ki67/PSA-ratio (P=0.001, n=29, FIG. 16B). This cut-off value was applicable also to the bone metastasis tissue cohort for dividing patients into two groups with long or short cancer-specific survival after ADT, respectively (FIG. 16C).

The absolute increased risk with increased Ki67/PSA-ratio for 1) death from prostate cancer if treated with active surveillance and 2) short time to progression if treated with ADT for metastatic prostate cancer need to be determined in prospective patient cohorts.

Table 16 illustrates the differences between different types of biopsies and disease stages. Illustrative cut offs are provided with reference to the tables/figures from which the values can be sourced. It should be understood that depending on the clinical situation, different cut-off could be chosen based on whether sensitivity or selectivity is prioritized.

TABLE 1
Top 60 differentiating gene transcripts per subtype.

| MetA-enriched | | MetB-enriched | | MetC-enriched | |
|---|---|---|---|---|---|
| Symbol | Gene ID | Symbol | Gene ID | Symbol | Gene ID |
| ACAA1 | ILMN_1738921 | ASPM | ILMN_1815184 | AEBP1 | ILMN_1736178 |
| ACP6 | ILMN_2234343 | BUB1 | ILMN_2202948 | AP1S2 | ILMN_2120273 |
| ACPP | ILMN_1758323 | C12orf48 | ILMN_1727055 | ARHGAP23 | ILMN_1764571 |
| ACSS1 | ILMN_1752269 | C16orf75 | ILMN_1790537 | ARHGEF6 | ILMN_1803423 |
| ALDH1A3 | ILMN_2139970 | C17orf53 | ILMN_1776490 | BMP1 | ILMN_1800412 |
| ALDH6A1 | ILMN_1785284 | C1orf135 | ILMN_1787280 | C10orf54 | ILMN_2205963 |
| ATP2C1 | ILMN_2340565 | C6orf173 | ILMN_1763907 | C1orf54 | ILMN_1702231 |
| C9orf91 | ILMN_1803652 | CCNA2 | ILMN_1786125 | C1QTNF5 | ILMN_1744487 |
| CANT1 | ILMN_1664012 | CCNB1 | ILMN_1712803 | CAV1 | ILMN_2149226 |
| CDH1 | ILMN_1770940 | CCNB2 | ILMN_1801939 | CD93 | ILMN_1704730 |
| CDS1 | ILMN_1801476 | CDC2 | ILMN_1747911 | CDH5 | ILMN_1719236 |
| COG3 | ILMN_1776154 | CDC20 | ILMN_1663390 | CLDN5 | ILMN_1728197 |
| CPNE4 | ILMN_1814770 | CDC45L | ILMN_1670238 | CLIP3 | ILMN_1789733 |
| CRELD1 | ILMN_1739558 | CDCA3 | ILMN_1737728 | COL6A2 | ILMN_1783909 |
| CTBS | ILMN_2144573 | CDCA4 | ILMN_1684045 | COL6A3 | ILMN_1706643 |
| DHRS7 | ILMN_1807455 | CENPF | ILMN_1664516 | COX7A1 | ILMN_1662419 |
| ENTPD5 | ILMN_1745849 | CENPL | ILMN_1742779 | CYYR1 | ILMN_1812902 |
| ENTPD6 | ILMN_2091792 | CKS1B | ILMN_1719256 | DDR2 | ILMN_2410523 |
| FAM174B | ILMN_1652797 | CKS2 | ILMN_2072296 | DPYSL2 | ILMN_1672503 |
| FICD | ILMN_1778064 | DDX39 | ILMN_1747303 | ENG | ILMN_1760778 |
| GABARAPL2 | ILMN_1796458 | DEK | ILMN_1747630 | FAM176B | ILMN_1769092 |
| GREB1 | ILMN_1721170 | ECT2 | ILMN_1717173 | FERMT2 | ILMN_1695290 |
| GTF3C1 | ILMN_1789839 | FAM83D | ILMN_1781943 | FGD5 | ILMN_2104141 |
| H2AFJ | ILMN_1708728 | GAS2L3 | ILMN_2211003 | FNDC1 | ILMN_1734653 |
| HPN | ILMN_1687235 | HMGB2 | ILMN_1654268 | FXYD5 | ILMN_2309848 |
| IVD | ILMN_1724207 | KIF11 | ILMN_1794539 | GAS6 | ILMN_1779558 |
| KIAA0251 | ILMN_1703969 | KIF15 | ILMN_1753063 | GIMAP4 | ILMN_1748473 |
| KLK2 | ILMN_2371917 | KIF20A | ILMN_1695658 | GIMAP8 | ILMN_1747305 |
| KLK3 | ILMN_1663787 | KIF23 | ILMN_1811472 | GJA4 | ILMN_1671106 |
| LOC124220 | ILMN_1753139 | KIFC1 | ILMN_2222008 | GYPC | ILMN_1668039 |
| LOC642299 | ILMN_1810431 | LIN9 | ILMN_2137084 | ICAM2 | ILMN_1786823 |
| LOC731999 | ILMN_1660277 | LOC399942 | ILMN_1765701 | IGFBP4 | ILMN_1665865 |
| NAAA | ILMN_1668605 | LOC643287 | ILMN_1677906 | ITGA5 | ILMN_1792679 |
| NECAB3 | ILMN_1749738 | LSM2 | ILMN_2070300 | JAM3 | ILMN_1769575 |
| NWD1 | ILMN_1721540 | MAD2L1 | ILMN_1777564 | KIAA1602 | ILMN_1763640 |
| PLA2G4F | ILMN_1744211 | MCM10 | ILMN_2413898 | LOC730994 | ILMN_1680774 |
| PPAP2A | ILMN_2343278 | MCM2 | ILMN_1681503 | LYL1 | ILMN_2216582 |
| PSD4 | ILMN_2154115 | MCM7 | ILMN_1663195 | MGC4677 | ILMN_2143795 |
| REXO2 | ILMN_1749009 | MDC1 | ILMN_1814122 | MSN | ILMN_1659895 |
| RNF41 | ILMN_1808095 | MEST | ILMN_1669479 | NAALADL1 | ILMN_1770963 |
| SC5DL | ILMN_1677607 | MSH6 | ILMN_1729051 | NINJ2 | ILMN_1731745 |
| SCCPDH | ILMN_1795839 | NCAPG | ILMN_1751444 | PARVG | ILMN_1695851 |
| SEC22C | ILMN_2290618 | NUSAP1 | ILMN_1726720 | PDGFRB | ILMN_1815057 |
| SEC23B | ILMN_2366246 | OIP5 | ILMN_2196984 | PECAM1 | ILMN_1689518 |
| SECISBP2L | ILMN_1784333 | PHF16 | ILMN_1790518 | PLCG2 | ILMN_1815719 |
| SELT | ILMN_1746368 | PSRC1 | ILMN_1671843 | PLCL2 | ILMN_1737025 |
| SLC25A17 | ILMN_1737312 | PTMA | ILMN_1759954 | RAB31 | ILMN_1660691 |
| SLC35A3 | ILMN_1653429 | PTTG3P | ILMN_2049021 | RASIP1 | ILMN_1755657 |
| SLC37A1 | ILMN_1687495 | RACGAP1 | ILMN_2077550 | SH2B3 | ILMN_1752046 |
| SLC39A6 | ILMN_1750394 | RFC5 | ILMN_1659364 | SH3KBP1 | ILMN_1810782 |
| SLC4A4 | ILMN_1734897 | STIL | ILMN_2413650 | SLIT3 | ILMN_1811313 |
| SLC9A2 | ILMN_1738849 | STMN1 | ILMN_1657796 | SRPX2 | ILMN_1676213 |
| SLC9A3R1 | ILMN_1680925 | TOP2A | ILMN_1686097 | STAB1 | ILMN_1655987 |
| STEAP2 | ILMN_2344298 | TPX2 | ILMN_1796949 | STOM | ILMN_1766657 |
| SUOX | ILMN_1803745 | TTK | ILMN_1788166 | TCF4 | ILMN_1814194 |
| TSPAN1 | ILMN_1747546 | TUBB | ILMN_2101885 | TEK | ILMN_2066151 |
| WASF3 | ILMN_1810797 | UBE2C | ILMN_2301083 | TPM2 | ILMN_1789196 |
| VIPR1 | ILMN_2199389 | UNG | ILMN_1683120 | TPST2 | ILMN_2329679 |
| VPS54 | ILMN_1761086 | USP1 | ILMN_1696975 | UBTD1 | ILMN_1794914 |
| XBP1 | ILMN_1809433 | ZNF250 | ILMN_1757230 | VAMP5 | ILMN_1809467 |

The term “Gene ID” refers to the Illumina BeadChips microarray probe accession number in the NCBI Probe database (www.ncbi.nlm.nih.gov/probe).

TABLE 2
Patient characteristics at prostate cancer diagnosis and at time for
metastasis surgery in relation to metastasis subtypes MetA-C^{a}.

| | MetA^{a} | | MetB^{a} | | MetC^{a} | |
|---|---|---|---|---|---|---|
| | n = 51 | | n = 12 | | n = 9 | |
| Age diagnosis (yrs) | 71 | (66; 76) | 64 | (59; 76) | 71 | (63; 76) |
| Age metastasis surgery (yrs) | 74 | (69; 80) | 68 | (62; 76)^{P=0.084} | 74 | (71; 79) |
| PSA diagnosis (ng/ml) | 160 | (58; 920) | 45 | (19; 76)* | 81 | (29; 130)^{P=0.075} |
| PSA metastasis surgery (ng/ml) | 470 | (110; 1100) | 84 | (44; 330)^{P=0.059} | 120 | (110; 180)^{P=0.068} |
| Follow-up from diagnosis (mo.) | 56 | (29; 84) | 30 | (24; 65) | 43 | (30; 110) |
| Follow up from first ADT^{b} (mo.) | 54 | (25; 78) | 30 | (21; 43) | 43 | (30; 98) |
| Follow up from metastasis surgery (mo.) | 10 | (3; 33) | 5 | (2; 11) | 13 | (5; 19) |
| Gleason score at diagnosis | | | | | | |
| 7 | 13 | (25%) | 3 | (25%) | 3 | (33%) |
| 8 | 13 | (25%) | 2 | (17%) | 4 | (44%) |
| 9 | 10 | (20%) | 3 | (25%) | 1 | (11%) |
| Not available | 15 | (29%) | 4 | (33%) | 1 | (11%) |
| Treatment with curative intention | | | | | | |
| Radical prostatectomy | 1 | (2%) | 0 | (0%) | 1 | (11%) |
| Radiation | 3 | (6%) | 4 | (33%)** | 1 | (11%) |
| Previous ADT^{b}: | | | | | | |
| None | 9 | (18%) | 1 | (8%) | 2 | (22%) |
| Short-term^{c} | 4 | (8%) | 0 | (0%) | 0 | (0%) |
| Long-term | 38 | (74%) | 11 | (92%) | 7 | (78%) |
| Additional therapies: | | | | | | |
| Bicalutamide | 17 | (33%) | 8 | (67%)* | 5 | (56%) |
| Chemotherapy | 4 | (8%) | 4 | (33%)* | 1 | (11%) |
| Ra223 | 3 | (6%) | 1 | (8%) | 1 | (11%) |
| Bisphosphonate | 5 | (10%) | 1 | (8%) | 1 | (11%) |
| Radiation towards operation site | 7 | (14%) | 1 | (8%) | 1 | (11%) |
| Soft tissue metastases | 9 | (18%) | 5 | (42%)^{P=0.072} | 1 | (11%)^{P=0.053} |
| Cancer cells^{d} (%) | 70 | (60; 80) | 70 | (70; 80) | 50 | (35; 50)** |

Continuous variables given as median (25th; 75th percentiles),
*P < 0.05;
**P < 0.01, compared to MetA.
^{a}Metastasis subtype, MetA-C, as determined from principal component analysis of whole genome expression profiles followed by unsupervised clustering (see materials and methods for details)
^{b}Androgen deprivation therapy (ADT) included surgical ablation or LHRH/GnRH agonist therapy.
^{c}ADT for 2-17 days before metastasis surgery.
^{d}Fraction of cancer cell content in frozen metastasis sections extracted for RNA and analyzed by whole genome expression analysis.

TABLE 3
Molecular metastasis subtypes MetA-C^{a} in relation
to metastasis and primary tumor morphology.

| | | |
|---|---|---|
| MetA | MetB | MetC |
| (n = 51) | (n = 12) | (n = 9) |

| | | | | | | | |
|---|---|---|---|---|---|---|---|
| Bone | AR score (0-12) | 8 | (4; 12) | 10 | (6; 12) | 9 | (4; 12) |

| | | | |
|---|---|---|---|
| metastases | (n = 49) | (n = 12) | (n = 8) |

| | | | | | | |
|---|---|---|---|---|---|---|
| PSA score (0-12) | 9 | (6; 12) | 2 | (1; 6)***a | 6 | (1; 9)a* |

| | | |
|---|---|---|
| (n = 51) | (n = 12) | (n = 9) |

| | | | | | | |
|---|---|---|---|---|---|---|
| Ki67 (%) | 14 | (9; 20) | 33 | (22; 45)***a | 12 | (8; 28)b* |

| | | |
|---|---|---|
| (n = 50) | (n = 12) | (n = 9) |

| | | | | | | |
|---|---|---|---|---|---|---|
| Chromogranin A (%) | 0 | (0; 0.2) | 0.2 | (0; 1.6)^{P=0.05}a | 0 | (0; 0) |

| | | | |
|---|---|---|---|
| | (n = 45) | (n = 12) | (n = 9) |
| Cellular atypia | 34; 17 | 2; 10**a | 2; 7*a |
| (moderate; high) | | | |
| Gland formation | 20; 31 | 0; 12**a | 4; 5*a |
| (yes; no) | | | |

| | | MetA | | MetB | | MetC | |
|---|---|---|---|---|---|---|---|
| | | associated | | associated | | associated | |
| | | (n = 36) | | (n = 8) | | (n = 8) | |
| Primary | AR score (0-12) | 12 | (12; 12) | 12 | (10; 12) | 10.5 | (8; 12) |
| tumor | | (n = 34) | | (n = 8) | | (n = 8) | |
| | PSA score (0-12) | 9 | (8; 12) | 6 | (4; 8)a** | 7 | (6; 10.5) |
| | | (n = 32) | | (n = 8) | | (n = 8) | |
| | Ki67 (%) | 9 | (6.5; 14) | 19 | (15; 26)a** | 17 | (11; 30)a* |
| | | (n = 35) | | (n = 8) | | (n = 8) | |
| | AR tumor stroma score | 22 | (15; 30) | 11 | (4; 17)a* | 17 | (7; 20) |

| | | | |
|---|---|---|---|
| (% of stroma cells | (n = 34) | (n = 8) | (n = 8) |
| positive) | | | |
| Reactive stroma score | 11; 11; 0 | 0; 2; 7***a | 0; 6; 1*a, b* |
| (1; 2; 3) | | | |

Continuous variables given as median (25th; 75th percentiles)
*P < 0.05,
***P < 0.00,
a = significantly different from MetA,
b = significantly different from MetB
^{a}Metastasis subtype, MetA-C, as determined from principal component analysis of whole genome expression profiles followed by unsupervised clustering (see FIG. 1)

TABLE 4
Multivariate Cox analysis of PSA and Ki67 immunoreactivity and
Gleason score (GS) in relation to cancer-specific survival of
patients diagnosed at TUR-P and managed by watchful-waiting.

| | | 95% CI | | |
|---|---|---|---|---|
| | HR | Lower | Upper | P |
| GS ≤ 6, n = 131 | 1 | | | |
| GS = 7, n = 47 | 3.8 | 1.8 | 8.1 | 4.8E−04 |
| GS ≥ 8, n = 59 | 6.7 | 3.2 | 14 | 4.9E−07 |
| PSA IR = 12, n = 132 | 1 | | | |
| PSA IR < 12, n = 105 | 2.1 | 1.1 | 3.7 | 0.017 |
| Ki67 (%) | 1.05 | 1.0 | 1.1 | 0.038 |

PSA immunoreactivity (IR) was dichotomized by the median value 9 as high (IR = 12) or low (≤9).
Fraction of Ki67 positive tumor cells was analyzed as a continuous variable.
CI = confidence interval.

TABLE 5
Clinical and histopathological variables in patients stratified by differences in Ki67 and PSA immunostaining.

| | | | |
|---|---|---|---|
| PSAhigh/Ki67low | PSAhigh/Ki67high | PSAlow/Ki67low | PSAlow/Ki67high |
| (n = 141, 42%) | (n = 17, 5%) | (n = 105, 32%) | (n = 68, 20%) |

Clinical markers

| Age | 74 | (69; 78) | 75 | (71; 79) | 74 | (69; 78)***a | 75 | (69; 82)***a, ***b |
|---|---|---|---|---|---|---|---|---|
| GS | | | | | | | | |
| 4-6 | 95 | (67) | 9 | (53) | 37 | (35) | 7 | (10) |
| 7 | 29 | (21) | 3 | (18) | 23 | (22) | 7 | (10) |
| 8-10 | 17 | (12) | 5 | (29)*a | 45 | (43)***a | 54 | (79)***a, **b |
| Tumor stage | | | | | | | | |
| T1 | 94 | (67) | 7 | (41) | 38 | (36) | 11 | (16) |
| T2 | 35 | (25) | 5 | (29) | 31 | (30) | 20 | (29) |
| T3-4 | 11 | (7.8) | 5 | (29) | 33 | (31) | 34 | (50) |
| x | 1 | (0.7) | 0*a | | 3 | (3)**a | 3 | (4)***a, **b |
| M stage | | | | | | | | |
| 0 | 100 | (71) | 11 | (65) | 73 | (70) | 35 | (51) |
| 1 | 3 | (2) | 2 | (12) | 13 | (12) | 23 | (34) |
| x | 38 | (27) | 4 | (24) | 19 | (18) | 10 | (15) |
| Cancer (%) | 10 | (7.5; 45) | 30 | (10; 70) | 60 | (20; 85)***a | 88 | (50; 95)***a, ***b |
| PC death (%) | 26 | (18) | 5 | (29) | 51 | (49)***a | 50 | (74)***a,**b |

Tumor markers

| pEGF-R score (36) | 3.1 | (2.4; 3.6) | 3.6 | (3.1; 3.9) | 3.3 | (2.8; 3.6) | 3.6 | (3.3; 4.0)***a, **b |
|---|---|---|---|---|---|---|---|---|
| (epithelial) | (n = 110) | | (n = 8) | | (n = 83) | | (n = 45) | |
| ErbB2 score (42) | 2.8 | (2.0; 3.0) | 3.0 | (2.7; 3.8)**a | 3.0 | (2.3; 3.8)**a | 3.0 | (3.0; 4.0)***a, *b |
| (epithelial) | (n = 126) | | (n = 14) | | (n = 99) | | (n = 63) | |
| ERG (43) | | | | | | | | |
| negative | 105 | (79.5) | 10 | (62.5) | 43 | (44.3) | 21 | (32.3) |
| positive | 27 | (20.5) | 6 | (37.5) | 54 | (55.7)***a | 44 | (67.7)***a |
| (epithelial) | | | | | | | | |
| pAkt score (44) | 2.6 | (2.2; 2.9) | 2.8 | (2.5; 3.3) | 2.8 | (2.4; 3.1)**a | 3.1 | (2.8; 3.6)***a, ***b |
| (epithelial) | (n = 109) | | (n = 12) | | (n = 81) | | (n = 49) | |
| Ki67 (%) (11, 12) | 1.4 | (0.4; 2.7) | 8.8 | (7.5; 13.6)***a, ***b | 2.5 | (1.2; 3.6)***a | 10.9 | (7.2; 15.6)***a, ***b |
| (epithelial) | (n = 141) | | (n = 17) | | (n = 105) | | (n = 68) | |
| Vascular density (%) | 11 | (8; 16) | 16 | (9; 19) | 15 | (10; 21)**a | 19 | (12; 24)***a,*b |
| (11, 12) | (n = 138) | | (n = 17) | | (n = 101) | | (n = 68) | |
| Hyaluronic acid score | 7.1 | (4.6; 9.0) | 9 | (6; 11)*a | 7.8 | (5.6; 9.8)*a | 8.6 | (6.2; 11.3)***a |
| (45) (stroma) | (n = 139) | | (n = 17) | | (n = 105) | | (n = 67) | |
| Mast cell density (%) | 13 | (9; 16) | 14 | (7; 17) | 12 | (8; 16) | 8 | (4; 13)***a,***b |
| (48) | (n = 134) | | (n = 16) | | (n = 100) | | (n = 65) | |
| Androgen receptor (%) | 50 | (39; 65) | 52 | (22; 67) | 48 | (28; 64) | 37 | (14; 55)***a,**b |
| (15) (stroma) | (n = 136) | | (n = 16) | | (n = 103) | | (n = 67) | |
| Caveolin-1 score | 3.0 | (2.8; 3.4) | 3.1 | (2.9, 3.4) | 3.0 | (2.8; 3.3)*a | 2.8 | (2.6; 3.1)***a, **b |
| (46) (stroma) | (n = 139) | | (n = 16) | | (n = 101) | | (n = 64) | |
| CD163 (%) | 16 | (11; 22) | 21 | (12; 30) | 19 | (16; 28)***a | 19 | (14; 26) |

| | | | | |
|---|---|---|---|---|
| (47) | (n = 87) | (n = 4) | (n = 53) | (n = 29) |

TINT markers

| Ki67 (%) (11, 12) | 0.2 | (0; 1.2) | 0 | (0; 1.3) | 0.3 | (0; 1.2) | 0.5 | (0; 2.5) |
|---|---|---|---|---|---|---|---|---|
| (epithelial) | (n = 138) | | (n = 17) | | (n = 95) | | (n = 57) | |
| pEGF-R score (36) | 3.0 | (1.8; 3.5) | 2.7 | (2.1; 3.5) | 3.3 | (2.5; 3.8)**a | 3.5 | (3; 3.9)**a |
| (epithelial) | (n = 111) | | (n = 9) | | (n = 79) | | (n = 40) | |
| pAKT score (44) | 2.0 | (1.5; 2.5) | 2.0 | (1.4; 2.3) | 2.3 | (1.6; 2.8) | 2.4 | (1.5; 2.8) |
| (epithelial) | (n = 92) | | (n = 11) | | (n = 56) | | (n = 34) | |
| ERG (43) | | | | | | | | |
| negative | 117 | (92.9) | 13 | (76.5) | 75 | (85.2) | 40 | (83.3) |
| positive | 9 | (7.1) | 4 | (23.5)*a | 13 | (14.8) | 8 | (16.7)*a |
| (epithelial) | | | | | | | | |
| Hyaluronic acid score | 6.3 | (4.3; 8.0) | 5.5 | (3.8; 8.1) | 6.5 | (5.0; 9.0) | 7 | (5; 9) |
| (45) (stroma) | (n = 135) | | (n = 17) | | (n = 99) | | (n = 59) | |
| Mast cell density (%) | 12 | (8; 15) | 12 | (9; 16) | 14 | (10; 20)**a | 14 | (11; 20)**a |

| | | | | |
|---|---|---|---|---|
| (48) | n = 130 | (n = 17) | (n = 91) | (n = 53) |

Continuous variables given as median (25^{th}; 75^{th} percentiles). Ordinal variables given as number (percentage).
x = unknown
a = significantly different from PSA high/Ki67 low
b = significantly different from PSA low/Ki67 low
Mann Whitney U test or Chi square test,
*p < 0.05,
**p < 0.01,
***p < 0.001

TABLE 6
Significant Spearman rank correlations between tumor PSA score
and Ki67 labeling index with other previously measured variables
of prognostic significance (see Table 5 for references) describing
tumor and surrounding normal prostate tissue (TINT).

| | |
|---|---|
| Correlation coefficient | Correlation coefficient |
| for tumor PSA score | for tumor Ki67 labeling index |

Clinical markers

| | | | | |
|---|---|---|---|---|
| Gleason score | −0.54*** | (n = 346) | 0.50*** | (n = 389) |
| Tumor stage | −0.41*** | (n = 339) | 0.42*** | (n = 382) |
| M stage | −0.31*** | (n = 272) | 0.33*** | (n = 301) |
| Cancer (%) | −0.47*** | (n = 346) | 0.45*** | (n = 389) |
| Overall survival | 0.21*** | (n = 346) | −0.15** | (n = 389) |

Tumor markers

| | | | | |
|---|---|---|---|---|
| Ki67 (%) | −0.46*** | (n = 331) | | |
| pEGF-R score | −0.21** | (n = 252) | 0.28*** | (n = 293) |
| pAkt score | −0.31*** | (n = 255) | 0.36*** | (n = 278) |
| ErbB2 score | −0.29*** | (n = 307) | 0.29*** | (n = 350) |
| Vascular density (%) | −0.24*** | (n = 330) | 0.28*** | (n = 381) |
| Hyaluronic acid score (stroma) | −0.18** | (n = 334) | 0.27*** | (n = 384) |
| Mast cell density (%) | 0.21*** | (n = 322) | −0.13* | (n = 362) |
| Androgen receptor (%) (stroma) | 0.17** | (n = 329) | −0.17** | (n = 373) |
| PDGFR-beta (stroma) (37) | −0.15* | (n = 248) | 0.21*** | (n = 283) |
| Caveolin-1 score (stroma) | 0.25*** | (n = 326) | −0.25*** | (n = 370) |
| CD163 (%) | −0.24** | (n = 177) | | |
| Erg (positive or not) | −0.39*** | (n = 315) | 0.32*** | (n = 350) |

TINT markers

| | | | | |
|---|---|---|---|---|
| Ki67 | | | 0.17** | (n = 360) |
| pEGF-R | −0.19** | (n = 244) | 0.25*** | (n = 284) |
| PDGFR-beta (stroma) (37) | −0.13* | (n = 302) | 0.15** | (n = 344) |
| Hyaluronic acid score (stroma) | −0.14* | (n = 318) | 0.14** | (n = 363) |
| Mast cell density (%) | −0.22*** | (n = 299) | | |
| Caveolin-1 score (stroma) | | | −0.11* | (n = 352) |
| Erg (positive or not) | | | 0.16** | (n = 331) |

*Correlation is significant at the <0.05 level (2-tailed)
**Correlation is significant at the <0.01 level (2-tailed)
***Correlation is significant at the <0.001 level (2-tailed)

TABLE 7
Sensitivity and specificity of Ki67 immunoreactivity (%) in primary
tumor biopsies for differentiating prostate cancer patients with low
to intermediate risk tumors from patients with progressive disease
(high risk, locally advanced, regionally metastasized or M1 tumors).

| Ki67 (%) | Sensitivity | 1 - Specificity |
|---|---|---|
| 1 | 1 | 0.889 |
| 2 | 1 | 0.689 |
| 3 | 1 | 0.578 |
| 4 | 0.969 | 0.4 |
| 5 | 0.938 | 0.356 |
| 6 | 0.908 | 0.267 |
| 7 | 0.785 | 0.222 |
| 8 | 0.723 | 0.156 |
| 9 | 0.677 | 0.133 |
| 10 | 0.615 | 0.133 |
| 11 | 0.554 | 0.111 |
| 12 | 0.538 | 0.044 |
| 13 | 0.477 | 0.044 |
| 14 | 0.431 | 0.044 |
| 15 | 0.4 | 0.022 |
| 16 | 0.323 | 0.022 |
| 17 | 0.308 | 0.022 |
| 18 | 0.292 | 0.022 |
| 19 | 0.277 | 0.022 |
| 20 | 0.246 | 0.022 |
| 21 | 0.215 | 0 |
| 22 | 0.2 | 0 |
| 23 | 0.154 | 0 |
| 26 | 0.123 | 0 |
| 29 | 0.108 | 0 |
| 31 | 0.092 | 0 |
| 33 | 0.062 | 0 |
| 36 | 0.046 | 0 |
| 39 | 0.031 | 0 |
| 42 | 0.015 | 0 |
| 44 | 0 | 0 |

TABLE 8
Sensitivity and specificity of PSA immunoreactivity score in primary
tumor biopsies for differentiating prostate cancer patients with low
to intermediate risk tumors from patients with progressive disease
(high risk, locally advanced, regionally metastasized or M1 tumors).

| PSA | Sensitivity | 1 - Specificity |
|---|---|---|
| 1 | 0 | 0 |
| 2 | 0.031 | 0 |
| 3 | 0.046 | 0 |
| 4 | 0.062 | 0 |
| 6 | 0.2 | 0.022 |
| 8 | 0.738 | 0.578 |
| 9 | 0.815 | 0.578 |
| 12 | 0.923 | 0.689 |

TABLE 9
Sensitivity and specificity of Ki67 immunoreactivity (%) in
primary tumor biopsies for differentiating prostate cancer
patients with short and long time to progression after androgen
deprivation therapy given to treat metastatic disease.

| Ki67 (%) | Sensitivity | 1 - Specificity |
|---|---|---|
| 2 | 1 | 1 |
| 6 | 1 | 0.867 |
| 7 | 0.857 | 0.8 |
| 8 | 0.857 | 0.733 |
| 9 | 0.857 | 0.667 |
| 11 | 0.786 | 0.533 |
| 12 | 0.786 | 0.467 |
| 14 | 0.714 | 0.467 |
| 15 | 0.714 | 0.4 |
| 17 | 0.571 | 0.333 |
| 19 | 0.571 | 0.267 |
| 20 | 0.5 | 0.267 |
| 21 | 0.429 | 0.267 |
| 22 | 0.357 | 0.267 |
| 26 | 0.286 | 0.2 |
| 31 | 0.286 | 0.133 |
| 33 | 0.214 | 0.067 |
| 36 | 0.143 | 0.067 |
| 39 | 0.143 | 0 |
| 42 | 0.071 | 0 |
| 44 | 0 | 0 |

TABLE 10
Sensitivity and specificity of PSA immunoreactivity score in
primary tumor biopsies for differentiating prostate cancer
patients with short and long time to progression after androgen
deprivation therapy given to treat metastatic disease.

| PSA | Sensitivity | 1 - Specificity |
|---|---|---|
| 1 | 0 | 0 |
| 2 | 0.143 | 0 |
| 3 | 0.214 | 0 |
| 6 | 0.286 | 0.133 |
| 8 | 0.5 | 0.2 |
| 9 | 0.786 | 0.6 |
| 12 | 0.929 | 0.867 |

TABLE 11
Sensitivity and specificity of the Ki67/PSA immunoreactivity
ratio* for identifying death from prostate cancer at
different cut-offs. Patients were diagnosed at TUR-P
(1975-1991) and managed by watchful waiting.

| Ki67/PSA | Sensitivity | 1 - Specificity | RR | P |
|---|---|---|---|---|
| 0 | 1 | 1 | | |
| 0.1031 | 0.954 | 0.568 | 11 | 0.000054 |
| 0.1211 | 0.938 | 0.541 | | |
| 0.1305 | 0.923 | 0.53 | | |
| 0.139 | 0.908 | 0.524 | | |
| 0.1602 | 0.892 | 0.459 | | |
| 0.1681 | 0.877 | 0.443 | | |
| 0.179 | 0.862 | 0.416 | | |
| 0.1941 | 0.846 | 0.378 | | |
| 0.204 | 0.831 | 0.362 | 6.3 | 2.00E−10 |
| 0.2075 | 0.815 | 0.357 | | |
| 0.2178 | 0.8 | 0.341 | | |
| 0.2215 | 0.785 | 0.33 | | |
| 0.2275 | 0.769 | 0.319 | | |
| 0.2276 | 0.754 | 0.319 | | |
| 0.228 | 0.738 | 0.319 | | |
| 0.23 | 0.723 | 0.314 | | |
| 0.2541 | 0.708 | 0.308 | | |
| 0.2664 | 0.692 | 0.292 | | |
| 0.2725 | 0.677 | 0.286 | | |
| 0.2795 | 0.662 | 0.276 | | |
| 0.2888 | 0.646 | 0.265 | | |
| 0.296 | 0.631 | 0.249 | | |
| 0.3167 | 0.615 | 0.222 | 3.9 | 1.41E−07 |
| 0.32 | 0.6 | 0.222 | | |
| 0.3389 | 0.585 | 0.211 | | |
| 0.3396 | 0.569 | 0.211 | | |
| 0.3416 | 0.554 | 0.211 | | |
| 0.3469 | 0.538 | 0.205 | | |
| 0.354 | 0.523 | 0.205 | | |
| 0.3602 | 0.508 | 0.205 | | |
| 0.37 | 0.492 | 0.2 | | |
| 0.383 | 0.477 | 0.184 | | |
| 0.3925 | 0.462 | 0.168 | | |
| 0.4042 | 0.446 | 0.168 | 3.3 | 0.000002 |
| 0.4186 | 0.431 | 0.162 | | |
| 0.4458 | 0.415 | 0.157 | | |
| 0.4739 | 0.4 | 0.146 | | |
| 0.5199 | 0.385 | 0.13 | 3.3 | 0.000005 |
| 0.607 | 0.369 | 0.097 | 3.8 | 2.35E−07 |
| 0.7851 | 0.354 | 0.076 | 3.8 | 3.36E−07 |
| 0.806 | 0.338 | 0.07 | 4.2 | 5.85E−08 |
| 0.8231 | 0.323 | 0.07 | | |
| 0.8475 | 0.308 | 0.07 | | |
| 0.8789 | 0.292 | 0.07 | | |
| 0.9028 | 0.277 | 0.07 | 4.0 | 0.000001 |
| 0.9158 | 0.262 | 0.07 | | |
| 0.9333 | 0.246 | 0.065 | | |
| 1.0675 | 0.231 | 0.049 | 4.1 | 0.000003 |
| 1.1758 | 0.215 | 0.038 | | |
| 1.2721 | 0.2 | 0.032 | 4.5 | 0.000003 |
| 1.3529 | 0.185 | 0.027 | | |
| 1.4493 | 0.169 | 0.027 | 6.5 | 2.17E−08 |
| 1.6262 | 0.154 | 0.022 | 11 | 2.28E−11 |
| 1.823 | 0.138 | 0.022 | 10 | 8.37E−10 |
| 2.0449 | 0.123 | 0.022 | 16 | 1.82E−11 |
| 2.3219 | 0.108 | 0.022 | | |
| 2.5111 | 0.092 | 0.022 | | |
| 2.6848 | 0.077 | 0.022 | | |
| 2.9484 | 0.062 | 0.022 | 16 | 7.39E−07 |
| 4.0147 | 0.046 | 0.016 | 11 | 0.00009 |
| 6.7258 | 0.031 | 0.011 | 9 | 0.002859 |
| 25.9191 | 0.015 | 0 | 18 | 0.00517 |
| 38.5408 | 0 | 0 | | |

(RR = relative risk value; P = probability value)
*The Ki67/PSA-ratio was obtained by dividing fraction of Ki67 positive tumor cells with the corresponding PSA immunoreactivity score.

TABLE 12
Cox regression analysis showing that the ratio between
Ki67 (%) and PSA immunoreactivity score in primary tumor
biopsies adds prognostic information to Gleason score
(GS), related to cancer-specific survival of patients
with prostate cancer managed by watchful-waiting.

| | P | RR |
|---|---|---|
| GS 4-6 | 0 | 1 |
| GS 7 | 0 | 4.57 |
| GS 8-10 | 0 | 8.626 |
| T1 | 0.196 | 1 |
| T2 | 0.128 | 1.638 |
| T3-4 | 0.097 | 1.846 |
| Ki67/PSA-ratio* | 0.007 | 1.072 |

(RR = relative risk value; P = probability value)
*The Ki67/PSA-ratio was obtained by dividing fraction of Ki67 positive tumor cells with the corresponding PSA immunoreactivity score.

TABLE 13
Sensitivity and specificity of the Ki67/PSA immunoreactivity
ratio* in primary tumor biopsies for differentiating prostate
cancer patients with low to intermediate risk tumors from
patients with progressive disease (high risk, locally advanced,
regionally metastasized or M1 tumors).

| Ki67/PSA | Sensitivity | 1 - Specificity |
|---|---|---|
| 0.0602 | 1 | 0.978 |
| 0.0735 | 1 | 0.956 |
| 0.0764 | 1 | 0.933 |
| 0.0806 | 1 | 0.911 |
| 0.0875 | 1 | 0.867 |
| 0.0927 | 1 | 0.844 |
| 0.1094 | 1 | 0.822 |
| 0.1312 | 1 | 0.8 |
| 0.1438 | 1 | 0.733 |
| 0.1583 | 1 | 0.711 |
| 0.1736 | 1 | 0.689 |
| 0.1861 | 1 | 0.667 |
| 0.2 | 1 | 0.644 |
| 0.2167 | 1 | 0.622 |
| 0.2375 | 1 | 0.6 |
| 0.3583 | 0.985 | 0.533 |
| 0.4313 | 0.969 | 0.422 |
| 0.4938 | 0.954 | 0.378 |
| 0.5125 | 0.923 | 0.378 |
| 0.5275 | 0.892 | 0.378 |
| 0.5483 | 0.877 | 0.378 |
| 0.5742 | 0.862 | 0.378 |
| 0.615 | 0.846 | 0.333 |
| 0.6483 | 0.831 | 0.333 |
| 0.7275 | 0.815 | 0.267 |
| 0.7428 | 0.8 | 0.267 |
| 0.7778 | 0.785 | 0.267 |
| 0.8188 | 0.769 | 0.267 |
| 0.8538 | 0.754 | 0.267 |
| 0.8725 | 0.738 | 0.267 |
| 0.8969 | 0.723 | 0.244 |
| 0.9583 | 0.708 | 0.2 |
| 0.9875 | 0.692 | 0.178 |
| 1.0278 | 0.677 | 0.178 |
| 1.1208 | 0.662 | 0.133 |
| 1.1438 | 0.646 | 0.133 |
| 1.1813 | 0.615 | 0.133 |
| 1.225 | 0.6 | 0.133 |
| 1.3125 | 0.569 | 0.111 |
| 1.4906 | 0.554 | 0.067 |
| 1.5625 | 0.538 | 0.067 |
| 1.6307 | 0.523 | 0.067 |
| 1.6515 | 0.508 | 0.067 |
| 1.7083 | 0.477 | 0.067 |
| 1.8438 | 0.462 | 0.044 |
| 1.9375 | 0.415 | 0.044 |
| 2.0625 | 0.4 | 0.044 |
| 2.1458 | 0.385 | 0.044 |
| 2.1896 | 0.369 | 0.044 |
| 2.2938 | 0.354 | 0.044 |
| 2.3875 | 0.338 | 0.044 |
| 2.45 | 0.323 | 0.044 |
| 2.6813 | 0.292 | 0.022 |
| 2.9375 | 0.277 | 0 |

*The Ki67/PSA-ratio was obtained by dividing fraction of Ki67 positive tumor cells with the corresponding PSA immunoreactivity score.

TABLE 14
Sensitivity and specificity of the Ki67/PSA immunoreactivity ratio* for
differentiation of patients with metastatic disease according to short
or long time to progression after androgen-deprivation therapy.

| Cut-off | Sensitivity | 1 - Specificity | RR | P |
|---|---|---|---|---|
| 0.3875 | 1 | 0.933 | — | NS |
| 0.6167 | 0.929 | 0.8 | — | NS |
| 1.0278 | 0.857 | 0.533 | — | NS |
| 1.8125 | 0.786 | 0.333 | 3.4 | 0.021 |
| 2.1875 | 0.643 | 0.267 | 2.1 | 0.003 |
| 2.625 | 0.571 | 0.267 | 3.3 | 0.011 |
| 2.9583 | 0.5 | 0.267 | 2.8 | 0.027 |
| 3.3333 | 0.429 | 0.267 | 3.3 | 0.05 |
| 4.7833 | 0.357 | 0.133 | — | NS |
| 6.35 | 0.286 | 0 | 5.2 | 0.006 |
| 6.75 | 0.214 | 0 | 18 | 0.002 |
| 23.5 | 0.143 | 0 | 18 | 0.004 |
| 41.5 | 0.071 | 0 | — | NS |
| 44 | 0 | 0 | | |

(RR = relative risk value; P = probability value)
*The Ki67/PSA-ratio was obtained by dividing fraction of Ki67 positive tumor cells with the corresponding PSA immunoreactivity score.

TABLE 15
Specific embodiments of the invention. Examples of combinations
of (i) the percentage of proliferation tumor cells; (ii) the PSA
immunoreactivity score; and (iii) the proliferation/PSA ratio.
When the PSA score is 0, the proliferation/PSA ratio has been
estimated to twice the percentage of proliferating tumor cells.

| | % proliferating tumor | | Proliferation/PSA |
|---|---|---|---|
| Embodiment# | cells | PSA score | ratio |
| 1 | 2 | 0 | 4.00 |
| 2 | 2 | 1 | 2.00 |
| 3 | 2 | 2 | 1.00 |
| 4 | 2 | 3 | 0.67 |
| 5 | 2 | 4 | 0.50 |
| 6 | 2 | 6 | 0.33 |
| 7 | 2 | 8 | 0.25 |
| 8 | 2 | 9 | 0.22 |
| 9 | 2 | 12 | 0.17 |
| 10 | 3 | 0 | 6.00 |
| 11 | 3 | 1 | 3.00 |
| 12 | 3 | 2 | 1.50 |
| 13 | 3 | 3 | 1.00 |
| 14 | 3 | 4 | 0.75 |
| 15 | 3 | 6 | 0.50 |
| 16 | 3 | 8 | 0.38 |
| 17 | 3 | 9 | 0.33 |
| 18 | 3 | 12 | 0.25 |
| 19 | 4 | 0 | 8.00 |
| 20 | 4 | 1 | 4.00 |
| 21 | 4 | 2 | 2.00 |
| 22 | 4 | 3 | 1.33 |
| 23 | 4 | 4 | 1.00 |
| 24 | 4 | 6 | 0.67 |
| 25 | 4 | 8 | 0.50 |
| 26 | 4 | 9 | 0.44 |
| 27 | 4 | 12 | 0.33 |
| 28 | 5 | 0 | 10.00 |
| 29 | 5 | 1 | 5.00 |
| 30 | 5 | 2 | 2.50 |
| 31 | 5 | 3 | 1.67 |
| 32 | 5 | 4 | 1.25 |
| 33 | 5 | 6 | 0.83 |
| 34 | 5 | 8 | 0.63 |
| 35 | 5 | 9 | 0.56 |
| 36 | 5 | 12 | 0.42 |
| 37 | 5.4 | 0 | 10.80 |
| 38 | 5.4 | 1 | 5.40 |
| 39 | 5.4 | 2 | 2.70 |
| 40 | 5.4 | 3 | 1.80 |
| 41 | 5.4 | 4 | 1.35 |
| 42 | 5.4 | 6 | 0.90 |
| 43 | 5.4 | 8 | 0.68 |
| 44 | 5.4 | 9 | 0.60 |
| 45 | 5.4 | 12 | 0.45 |
| 46 | 6 | 0 | 12.00 |
| 47 | 6 | 1 | 6.00 |
| 48 | 6 | 2 | 3.00 |
| 49 | 6 | 3 | 2.00 |
| 50 | 6 | 4 | 1.50 |
| 51 | 6 | 6 | 1.00 |
| 52 | 6 | 8 | 0.75 |
| 53 | 6 | 9 | 0.67 |
| 54 | 6 | 12 | 0.50 |
| 55 | 7 | 0 | 14.00 |
| 56 | 7 | 1 | 7.00 |
| 57 | 7 | 2 | 3.50 |
| 58 | 7 | 3 | 2.33 |
| 59 | 7 | 4 | 1.75 |
| 60 | 7 | 6 | 1.17 |
| 61 | 7 | 8 | 0.88 |
| 62 | 7 | 9 | 0.78 |
| 63 | 7 | 12 | 0.58 |
| 64 | 10 | 0 | 20.00 |
| 65 | 10 | 1 | 10.00 |
| 66 | 10 | 2 | 5.00 |
| 67 | 10 | 3 | 3.33 |
| 68 | 10 | 4 | 2.50 |
| 69 | 10 | 6 | 1.67 |
| 70 | 10 | 8 | 1.25 |
| 71 | 10 | 9 | 1.11 |
| 72 | 10 | 12 | 0.83 |
| 73 | 16 | 0 | 32.00 |
| 74 | 16 | 1 | 16.00 |
| 75 | 16 | 2 | 8.00 |
| 76 | 16 | 3 | 5.33 |
| 77 | 16 | 4 | 4.00 |
| 78 | 16 | 6 | 2.67 |
| 79 | 16 | 8 | 2.00 |
| 80 | 16 | 9 | 1.78 |
| 81 | 16 | 12 | 1.33 |
| 82 | 20 | 0 | 40.00 |
| 83 | 20 | 1 | 20.00 |
| 84 | 20 | 2 | 10.00 |
| 85 | 20 | 3 | 6.67 |
| 86 | 20 | 4 | 5.00 |
| 87 | 20 | 6 | 3.33 |
| 88 | 20 | 8 | 2.50 |
| 89 | 20 | 9 | 2.22 |
| 90 | 20 | 12 | 1.67 |
| 91 | 25 | 0 | 50.00 |
| 92 | 25 | 1 | 25.00 |
| 93 | 25 | 2 | 12.50 |
| 94 | 25 | 3 | 8.33 |
| 95 | 25 | 4 | 6.25 |
| 96 | 25 | 6 | 4.17 |
| 97 | 25 | 8 | 3.13 |
| 98 | 25 | 9 | 2.78 |
| 99 | 25 | 12 | 2.08 |

TABLE 16
Exemplary cut off values

| Biopsy | Metastatic | Proliferation | PSA | Combination |
|---|---|---|---|---|
| type | disease | cut-off | cut-off | score cut off |
| Primary | No/unknown | 5% (Table 7) | 9 (Table 8) | 0.2 (Table 13) |
| tumor | | | | |
| Primary | Yes | 16% (Table 9) | 9 (Table 10) | 2.1 (Table 14) |
| tumor | | | | |
| Metastasis | Yes | 25% (FIG. 5) | 8 (FIG. 5) | 2.1 (Table 14) |

### REFERENCES

1. Omlin A, Pezaro C, Gillessen Sommer S. Sequential use of novel therapeutics in advanced prostate cancer following docetaxel chemotherapy. Ther Adv Urol 2014; 6:3-14.
2. Van Etten J L, Dehm S M. Clonal origin and spread of metastatic prostate cancer. Endocr Relat Cancer. 2016; 23:R207-17.
3. Bergstrom S H, Rudolfsson S H, Bergh A. Rat Prostate Tumor Cells Progress in the Bone Microenvironment to a Highly Aggressive Phenotype. Neoplasia 2016; 18:152-61.
4. Hörnberg E, Ylitalo E B, Crnalic S, et al. Expression of androgen receptor splice variants in prostate cancer bone metastases is associated with castration-resistance and short survival. PLoS One 2011; 6:e19059.
5. Jernberg E, Thysell E, Bovinder Ylitalo E, et al. Characterization of prostate cancer bone metastases according to expression levels of steroidogenic enzymes and androgen receptor splice variants. PLoS One 2013; 8:e77407.
6. Djusberg E, Jernberg E, Thysell E, et al. High levels of the AR-V7 Splice Variant and CoAmplification of the Golgi Protein Coding YIPF6 in A R Amplified Prostate Cancer Bone Metastases. Prostate 2017; 77:625-38.
7. Ylitalo E B, Thysell E, Jernberg E, et al. Subgroups of Castration-resistant Prostate Cancer Bone Metastases Defined Through an Inverse Relationship Between Androgen Receptor Activity and Immune Response. Eur Urol 2017; 71:776-87.7.
8. Nordstrand A, Bovinder Ylitalo E, Thysell E, et al. Bone Cell Activity in Clinical Prostate Cancer Bone Metastasis and Its Inverse Relation to Tumor Cell Androgen Receptor Activity. Int J Mol Sci 2018; 19.
9. Iglesias-Gato D, Thysell E, Tyanova S, Crnalic S, Santos A., Lima T S, Geiger T., Cox J, Widmark A, Bergh A, Mann M, Flores-Morales A., Wikström P. The proteome of prostate cancer bone metastasis reveals phenotypic heterogeneity with prognostic and therapeutic implications. Clin Cancer Res 2018 Jul. 24. pii: clincanres.1229.2018. doi: 10.1158/1078-0432.CCR-18-1229. [Epub ahead of print]
10. Crnalic S, Hornberg E, Wikström P, et al. Nuclear androgen receptor staining in bone metastases is related to a poor outcome in prostate cancer patients. Endocr Relat Cancer 2010; 17:885-95.
11. Josefsson A, Wikström P, Egevad L, et al. Low endoglin vascular density and Ki67 index in Gleason score 6 tumours may identify prostate cancer patients suitable for surveillance. Scand J Urol 2012; 46:247-57.
12. Josefsson A, Wikström P, Granfors T, et al. Tumor size, vascular density and proliferation as prognostic markers in G S 6 and G S 7 prostate tumors in patients with long follow-up and non-curative treatment. Eur Urol 2005; 48:577-83.
13. Frey B J, Dueck D. Clustering by passing messages between data points. Science 2007; 315:972-6.
14. Halin S, Hammarsten P, Wikström P, et al. Androgen-insensitive prostate cancer cells transiently respond to castration treatment when growing in an androgen-dependent prostate environment. Prostate 2007; 67:370-7.
15. Wikström P, Marusic J, Stattin P, et al. Low stroma androgen receptor level in normal and tumor prostate tissue is related to poor outcome in prostate cancer patients. Prostate 2009; 69:799-809.
16. Saeter T, Vlatkovic L, Waaler G, et al. The prognostic value of reactive stroma on prostate needle biopsy: a population-based study. Prostate 2015; 75:662-71.
17. Wierstra I, Alves J. FOXM1, a typical proliferation-associated transcription factor. Biol Chem 2007; 388:1257-74.
18. You S, Knudsen B S, Erho N, et al. Integrated Classification of Prostate Cancer Reveals a Novel Luminal Subtype with Poor Outcome. Cancer Res 2016; 76:4948-58.
19. Zhao S G, Chang S L, Erho N, et al. Associations of Luminal and Basal Subtyping of Prostate Cancer With Prognosis and Response to Androgen Deprivation Therapy. JAMA Oncol 2017; 3:1663-72.
20. Flores-Morales A, Bergmann T B, Lavallee C, et al. Proteogenomic characterization of patient-derived xenografts highlights the role of REST in neuroendocrine differentiation of castration-resistant prostate cancer. Clin Cancer Res. 2018 Oct. 1. pii: clincanres.0729.2018. doi:10.1158/1078-0432.CCR-18-0729. [Epub ahead of print].
21. Cai C, He H H, Chen S, et al. Androgen receptor gene expression in prostate cancer is directly suppressed by the androgen receptor through recruitment of lysine-specific demethylase 1. Cancer cell 2011; 20:457-71.
22. Gao S, Gao Y, He H H, et al. Androgen Receptor Tumor Suppressor Function Is Mediated by Recruitment of Retinoblastoma Protein. Cell Rep 2016; 17:966-76.
23. Yang S, Jiang M, Grabowska M M, et al. Androgen receptor differentially regulates the proliferation of prostatic epithelial cells in vitro and in vivo. Oncotarget 2016; 7:70404-19.
24. Sharma N L, Massie C E, Ramos-Montoya A, et al. The androgen receptor induces a distinct transcriptional program in castration-resistant prostate cancer in man. Cancer cell 2013; 23:35-47.
25. Liu S, Kumari S, Hu Q, et al. A comprehensive analysis of coregulator recruitment, androgen receptor function and gene expression in prostate cancer. eLife 2017; 6.
26. Gao N, Zhang J, Rao M A, et al. The role of hepatocyte nuclear factor-3 alpha (Forkhead Box A1) and androgen receptor in transcriptional regulation of prostatic genes. Mol Endocrinol 2003; 17:1484-507.
27. Ketola K, Munuganti RSN, Davies A, et al. Targeting Prostate Cancer Subtype 1 by Forkhead Box M1 Pathway Inhibition. Clin Cancer Res 2017; 23:6923-33.
28. Horning A M, Wang Y, Lin C K, et al. Single-Cell RNA-seq Reveals a Subpopulation of Prostate Cancer Cells with Enhanced Cell-Cycle-Related Transcription and Attenuated Androgen Response. Cancer Res 2018; 78:853-64.28. Cheville J C, Tindall D, Boelter C, et al. Metastatic prostate carcinoma to bone: clinical and pathologic features associated with cancer-specific survival. Cancer 2002; 95:1028-36.
29. Barakat D J, Zhang J, Barberi T, et al. CCAAT/Enhancer binding protein beta controls androgen-deprivation-induced senescence in prostate cancer cells. Oncogene 2015; 34:5912-22.
30. Duffy M J, Harbeck N, Nap M, et al. Clinical use of biomarkers in breast cancer: Updated guidelines from the European Group on Tumor Markers (EGTM). Eur J Cancer 2017; 75:284-98.
31. Bubendorf L, Tapia C, Gasser T C, et al. Ki67 labeling index in core needle biopsies independently predicts tumor-specific survival in prostate cancer. Hum Pathol 1998; 29:949-54.
32. Stege R, Grande M, Carlstrom K, et al. Prognostic significance of tissue prostate-specific antigen in endocrine-treated prostate carcinomas. Clin Cancer Res 2000; 6:160-5.
33. Fisher G, Yang Z H, Kudahetti S, et al. Prognostic value of Ki-67 for prostate cancer death in a conservatively managed cohort. Br J Cancer 2013; 108:271-7.
34. Brechbuhl H M, Finlay-Schultz J, Yamamoto T M, et al. Fibroblast Subtypes Regulate Responsiveness of Luminal Breast Cancer to Estrogen. Clin Cancer Res 2017; 23:1710-21.
35. Roswall P, Bocci M, Bartoschek M, et al. Microenvironmental control of breast cancer subtype elicited through paracrine platelet-derived growth factor-C C signaling. Nature Med 2018; 24:463-73.
36. Hammarsten P, Karalija A, Josefsson A, et al. Low levels of phosphorylated epidermal growth factor receptor in nonmalignant and malignant prostate tissue predict favorable outcome in prostate cancer patients. Clin Cancer Res 2010; 16:1245-55.
37. Egevad L, Granfors T, Karlberg L, et al. Prognostic value of the Gleason score in prostate cancer. BJU Int. 2002; 89:538-42.
38. van Leenders G J, Boormans J L, Vissers C J, et al. Antibody EPR3864 is specific for ERG genomic fusions in prostate cancer: implications for pathological practice. Mod Pathol 2011; 24:1128-38.
39. Barron D A, Rowley D R. The reactive stroma microenvironment and prostate cancer progression. Endocr Relat Cancer 2012; 19:R187-204.
40. Hägglöf C, Bergh A. The stroma-a key regulator in prostate function and malignancy. Cancers 2012; 4:531-48.
41. Adamo H H, Stromvall K, Nilsson M, et al. Adaptive (TINT) Changes in the Tumor Bearing Organ Are Related to Prostate Tumor Size and Aggressiveness. PLoS One 2015; 10:e0141601.
42. Hammarsten P, Winther J, Rudolfsson S H, et al. ErbB2 receptor immunoreactivity in prostate cancer: relationship to the androgen receptor, disease severity at diagnosis and disease outcome. PLoS One 2014; 9:e105063.
43. Hägglöf C, Hammarsten P, Stromvall K, et al. TMPRSS2-ERG expression predicts prostate cancer survival and associates with stromal biomarkers. PLoS One 2014; 9:e86824.35.
44. Hammarsten P, Cipriano M, Josefsson A, et al. Phospho-Akt immunoreactivity in prostate cancer: relationship to disease severity and outcome, Ki67 and phosphorylated EGFR expression. PLoS One 2012; 7:e47994.
45. Josefsson A, Adamo H, Hammarsten P, et al. Prostate cancer increases hyaluronan in surrounding nonmalignant stroma, and this response is associated with tumor growth and an unfavorable outcome. Am J Pathol 2011; 179:1961-8.
46. Hammarsten P, Dahl Scherdin T, Hägglöf C, et al. High Caveolin-1 Expression in Tumor Stroma Is Associated with a Favourable Outcome in Prostate Cancer Patients Managed by Watchful Waiting. PLoS One 2016; 11:e0164016.
47. Lundholm M, Hägglöf C, Wikberg M L, et al. Secreted Factors from Colorectal and Prostate Cancer Cells Skew the Immune Response in Opposite Directions. Sci Rep 2015; 5:15651.
48. Johansson A, Rudolfsson S, Hammarsten P, et al. Mast cells are novel independent prognostic markers in prostate cancer and represent a target for therapy. Am J Pathol 2010; 177:1031-41.
49. Bylesjo, M., et al. OPLS discriminant analysis: combining the strengths of PLS-D A and SIMCA classification. J Chemometr 2006; 20:341-351.
50. Quigley D A, Dang H X, Zhao S G, et al. Genomic Hallmarks and Structural Variation in Metastatic Prostate Cancer. Cell. 2018; 175:889.
51. Loeb S, Ross A E. Genomic testing for localized prostate cancer: where do we go from here? Curr Opin Urol. 2017 September; 27(5):495-499.

1. A method for determining tumor aggressiveness in a subject diagnosed with prostate cancer and having a tumor, said method comprising:
(a) evaluating the level of cell differentiation in a sample comprising tumor-derived material from the said subject; and
(b) evaluating the level of proliferating cells in the said sample;
wherein
(i) a low level of cell differentiation and a high level of proliferating cells are associated with high tumor aggressiveness, and
(ii) a high level of cell differentiation and a low level of proliferating cells are associated with low or moderate tumor aggressiveness.

2. The method according to claim 1, comprising:
(a) evaluating the level of cell differentiation in a sample comprising tumor-derived material from the said subject;
(b) evaluating the level of proliferating cells in the said sample; and
(c) deciding reference cut-off values for the level of cell differentiation and the level of proliferating cells;
wherein
(i) a lower level of cell differentiation compared to the corresponding reference cut-off value and a higher level of proliferating cells compared to the corresponding reference cut-off value are associated with high tumor aggressiveness, and
(ii) a higher level of cell differentiation compared to the corresponding reference cut-off value and a lower level of proliferating cells compared to the corresponding reference cut-off value are associated with low or moderate tumor aggressiveness.

3. The method according to claim 1 wherein evaluating the level of cell differentiation comprises evaluating the level of prostate-specific antigen (PSA).

4. The method according to claim 3 wherein evaluating the level of PSA in the sample comprises evaluating PSA immunoreactivity.

5. The method according to claim 1 wherein evaluating the level of proliferating cells in the sample comprises evaluating the fraction of Ki67, PCNA or MCM positive cells.

6. The method according to claim 5 wherein evaluating the fraction of Ki67 positive cells in the sample comprises evaluating Ki67 immunoreactivity.

7. The method according to claim 3, further comprising deriving a proliferation-PSA combination score from the proliferation/PSA ratio, said proliferation/PSA ratio being calculated by dividing the fraction of proliferating tumor cells in the sample multiplied with 100 with the PSA immunoreactivity score in the sample.

8. The method according to claim 7, wherein
(i) a high proliferation-PSA combination score is associated with high tumor aggressiveness, and
(ii) a low proliferation-PSA combination score is associated with low or moderate tumor aggressiveness.

9. The method according to claim 1 wherein the said tumor-derived material is obtained from a primary tumor.

10. The method according to claim 9 for determining the metastatic potential of a primary tumor, wherein high aggressiveness is associated with high metastatic potential and low aggressiveness is associated with low metastatic potential.

11. The method according to claim 9 wherein a PSA immunoreactivity score of 9 or lower, is associated with high tumor aggressiveness.

12. The method according to claim 11 wherein a fraction of 3% or more proliferating cells is associated with high tumor aggressiveness.

13. The method according to claim 9 wherein a fraction of 5.4% or more proliferating cells in combination with a PSA immunoreactivity score 9 or lower is associated with high tumor aggressiveness.

14-15. (canceled)

16. The method according to claim 1, wherein the subject has a metastasis.

17. The method according to claim 16 wherein a PSA immunoreactivity score of 9 or lower is associated with high tumor aggressiveness.

18. The method according to claim 16 wherein a fraction of 6% or more proliferating cells is associated with high tumor aggressiveness.

19. The method according to claim 16, wherein a fraction of 16% or more proliferating cells in combination with a PSA immunoreactivity score 9 or lower is associated with high tumor aggressiveness.

20-22. (canceled)

23. A method of treating prostate cancer in a subject in need thereof, said method comprising:
(a) using the method of claim 1 for determining tumor aggressiveness in a subject diagnosed with prostate cancer and having a metastasis, and
(b) administering a prostate cancer treatment to the subject; wherein
(i) if the tumor aggressiveness is low, the subject is administered androgen deprivation therapy and/or androgen receptor targeting therapy; and
(ii) if the tumor aggressiveness high, the subject is administered (I) androgen deprivation therapy and/or androgen receptor targeting therapy, in combination with (II) chemotherapy and/or therapy using DNA repair inhibitors.

24-32. (canceled)

33. A diagnostic method for classifying a prostate cancer subtype in a sample, said method comprising:
(a) obtaining a sample comprising tumor-derived material from a subject diagnosed with prostate cancer;
(b) obtaining a gene expression profile for the said test sample;
(c) comparing the obtained gene expression profile with the gene expression profile from a reference population;
(d) assigning the test sample to the prostate cancer subtype designated
(i) MetA, characterized by increased expression of at least 10 of the genes selected from the group consisting of ACAA1, ACP6, ACPP, ACSS1, ALDHIA3, ALDH6A1, ATP2C1, C9orf91, CANT1, CDH1, CDS1, COG3, CPNE4, CRELD1, CTBS, DHRS7, ENTPD5, ENTPD6, FAM174B, FICD, GABARAPL2, GREB1, GTF3C1, H2AFJ, HPN, IVD, KIAA0251, KLK2, KLK3, LOC124220, LOC642299, LOC731999, NAAA, NECAB3, NWD1, PLA2G4F, PPAP2A, PSD4, REXO2, RNF41, SCFDL, SCCPDH, SEC22C, SEC23B, SECISBP2L, SELT, SLC25A17, SLC35A3, SLC37A1, SLC39A6, SLC4A4, SLC9A2, SLC9A3R1, STEAP2, SUOX, TSPAN1, WASF3, VIPR1, VPS54, and XBP1;
(ii) MetB, characterized by increased expression of at least 10 of the genes selected from the group consisting of ASPM, BUB1, C12orf48, C16orf75, C17orf53, C1orf135, C6orf173, CCNA2, CCNB1, CCNB2, CDC2, CDC20, CDC451, CDCA3, CDCA4, CENPF, CENPL, CKS1B, CKS2, DDX39, DEK, ECT2, FAM83D, GAS2L3, HMGB2, KIF11, KIF15, KIF20A, KIF23, KIFC1, LIN9, LOC399942, LOC643287, LSM2, MAD2L1, MCM10, MCM2, MCM1, MDC1, MEST, MSH6, NCAPG, NUSAP1, OIP5, PHFI6, PSRC1, PTMA, PTTG3P, RACGAP1, RFCS, STIL, STMN1, TOP2A, TPX2, TTK, TUBB, UBE2C, UNG, USP1, and ZNF250; or
(iii) MetC, characterized by increased expression of at least 10 of the genes selected from the group consisting of AEBP1, APIS2, ARHGAP23, ARHGEF6, BMP1, C1orf54, C1orf54, C1QT1VF5, CAV1, CD93, CDH5, CLDN5, CLIP3, COL6A2, COL6A3, COX7A1, CYYR1, DDR2, DPYSL2, ENG, FAM176B, FERMT2, FGD5, FNDC1, FXYD5, GAS6, GIMAP4, GIMAP8, GJA4, GYPC, ICAM2, IGFBP4, ITGA5, JAM3, KIAA1602, LOC730994, LYL1, MGC4677, MSN, NAALADL1, NINJ2, PARVG, PDGFRB, PECAM1, PLCG2, PLCL2, RAB31, RASIP1, SH2B3, SH3KBP1, SLIT3, SRPX2, STAB1, STOM, TCF4, TEK, TPM2, TPST2, UBTD1, and VAMP5.
